(19) United States
(12) Patent Application Publication
(10) Pub. No.: US 2026/0217819 A1
(43) Pub. Date: Jul. 30, 2026

(54) ANTIBODIES, ANTIGEN-BINDING FRAGMENTS AND METHODS OF USE

(71) Applicants: HANSOH BIO LLC, Rockville, MD (US); SHANGHAI HANSOH BIOMEDICAL CO., LTD., Shanghai (CN); CHANGZHOU HANSOH PHARMACEUTICAL CO., LTD., Changzhou, Jiangsu (CN)

(72) Inventors: Qing LI, Rockville, MD (US); Ningyun ZHOU, Rockville, MD (US)

(73) Assignees: HANSOH BIO LLC, Rockville, MD (US); SHANGHAI HANSOH BIOMEDICAL CO., LTD., Shanghai (CN); CHANGZHOU HANSOH PHARMACEUTICAL CO., LTD., Changzhou, Jiangsu (CN)

(21) Appl. No.: 19/158,228

(22) PCT Filed: Feb. 23, 2024
(86) PCT No.: PCT/CN2024/078324

§ 371 Date: Aug. 20, 2025

Related U.S. Application Data
(60) Provisional application No. 63/486,522, filed on Feb. 23, 2023.

(51) Int. Cl. C07K 16/28; A61K 47/68; G01N 33/575

(52) U.S. Cl. CPC C07K 16/28; A61K 47/6849; G01N 33/5758; C07K 2317/56; C07K 2317/565; C07K 2317/77; C07K 2317/92; G01N 2333/705

(57) ABSTRACT

Provided are antibodies or antigen-binding fragments that specifically recognize human liver-intestine cadherin, and the pharmaceutical use thereof. The antibody or antigen-binding fragment containing specific amino acid sequences. The antibody or the antigen-binding fragment thereof has high specificity and affinity to liver-intestine cadherin, and can mediate the killing of liver-intestine cadherin expressing cells. Therefore, further relates to a pharmaceutical composition comprising the antibody or the antigen-binding fragment thereof, and use thereof in the preparation of medicament for the prevention and/or treatment of tumors.

The present disclosure claims the priority of U.S. Patent Application No. 63/486,522 filed on Feb. 23, 2023, which is incorporated herein by reference in its entirety.

### FIELD OF THE INVENTION

The present disclosure relates to antibodies, and in particular, to antibodies exhibiting specificity for liver-intestine cadherin, and to use thereof, for example in the treatment of cancer.

### BACKGROUND ART

Liver-intestine cadherin (Cadherin 17) is a cell surface marker belonging to cadherin superfamily with a unique biological structure. It has 7 extracellular cadherin repeats compared to the classic 5 cadherin repeats, and has a very short intracellular region of 20 amino acid residues lacking the conserved intracellular domain. (Berndorff et al. J Cell Biol. 1994, 125(6):1353-1369). Although the biological function of Cadherin 17 has not been fully explored yet, it has been reported that Cadherin 17 can regulate the water resorption in a Ca²⁺-dependent manner (Ahl et al. Biol. Med. Model. 2011, 8(18)). Cadherin 17 also involves in maintaining the tissue integrity by interacting with integrin within the extracellular tight junctions. It mainly expresses in human gastrointestinal (GI) tract and pancreas.

Cadherin 17 has been reported to be highly expressed in tumors including colorectal, gastric, and pancreatic tumors in both DNA and protein level (Takamura et al. Med Mol Morphol. 2013, 46:1-7). It plays an important role in regulating the cancer metastasis and tumor growth. There are several signaling pathways involved in the Cadherin 17 manipulated tumor activities. One of the most important mechanisms is related to Cadherin 17-intergin interaction. It has been proved that the RGD motif of Cadherin 17 binds to α2β1 integrin which induces the β1 integrin activation leading to increase cancer cell proliferation and adhesion (Bartomome et al. J Biol Chem. 2014, 289(50):34801-34914). It has also been shown that Cadherin 17 regulates the cancer invasion through Wnt/β-catenin signaling (Qiu et al. PloS one 2013, 8(3)) and NFκB signaling pathway (Wang et al. Cancer biology & therapy. 2013, 14(3): 262-270) in GI cancers. The restricted expression in normal tissue and high expression in various cancers make Cadherin 17 a good cancer target.

There are several Cadherin 17 targeted antibody-based drugs that have been studied in the field: two bispecific antibody drugs—B1905711 (Boehringer Ingelheim) and ARB202 (Arbele), anti-Cadherin 17 based CAR therapies and one monoclonal antibody drug: PA-0661 (ProAlt). PA-00661 is the only reported monoclonal antibody therapy that specifically detects Cadherin 17 RGD motif and it is currently in preclinical trails. Thus, there remains a need to develop a new anti-Cadherin 17 antibody to better treat cancers that have high Cadherin 17 expression.

### SUMMARY OF THE INVENTION

An object of the present disclosure is to provide a novel antibody or an antigen-binding fragment of the antibody (hereinafter, also referred to as an antibody, etc.) which binds to Cadherin 17, also provides a molecule comprising the antibody, and a pharmaceutical composition comprising the antibody, the antigen-binding fragment thereof or the molecule as an active ingredient.

The present inventors conducted extensive research to achieve the object mentioned above and have realized the present disclosure by developing a novel anti-Cadherin 17 antibody and a molecule comprising the same.

Specifically, the present disclosure encompasses at least the following aspects:

The present disclosure provides an anti-Cadherin 17 antibody or an antigen-binding fragment thereof comprises: a heavy chain variable region comprising HCDR1, HCDR2 and HCDR3 regions and a light chain variable region comprising LCDR1, LCDR2 and LCDR3 regions, wherein:
a) HCDR1 having the amino acid sequence as shown in SEQ ID NO: 01, SEQ ID NO: 02, SEQ ID NO: 03, SEQ ID NO: 04, SEQ ID NO: 05, SEQ ID NO: 06, SEQ ID NO: 07, SEQ ID NO: 08, SEQ ID NO: 09 or SEQ ID NO: 10;
b) HCDR2 having the amino acid sequence as shown in SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20 or SEQ ID NO: 21;
c) HCDR3 having the amino acid sequence as shown in SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30 or SEQ ID NO: 31;
d) LCDR1 having the amino acid sequence as shown in SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41 or SEQ ID NO: 42;
e) LCDR2 having the amino acid sequence as shown in SEQ ID NO: 43, SEQ ID NO: 44, SEQ ID NO: 45, SEQ ID NO: 46, or SEQ ID NO: 47;
f) LCDR3 having the amino acid sequence as shown in SEQ ID NO: 48, SEQ ID NO: 49, SEQ ID NO: 50, SEQ ID NO: 51, SEQ ID NO: 52, SEQ ID NO: 53 or SEQ ID NO: 54.

In some embodiments, the heavy chain variable region sequence comprises:
HCDR1 having the amino acid sequence as shown in SEQ ID NO: 01, HCDR2 having the amino acid sequence as shown in SEQ ID NO: 11, and HCDR3 having the amino acid sequence as shown in SEQ ID NO: 22, respectively; or
HCDR1 having the amino acid sequence as shown in SEQ ID NO: 02, HCDR2 having the amino acid sequence as shown in SEQ ID NO: 12, and HCDR3 having the amino acid sequence as shown in SEQ ID NO: 23 respectively; or
HCDR1 having the amino acid sequence as shown in SEQ ID NO: 03, HCDR2 having the amino acid sequence as shown in SEQ ID NO: 13, and HCDR3 having the amino acid sequence as shown in SEQ ID NO: 24 respectively; or
HCDR1 having the amino acid sequence as shown in SEQ ID NO: 04, HCDR2 having the amino acid sequence as shown in SEQ ID NO: 14, and HCDR3 having the amino acid sequence as shown in SEQ ID NO: 25 respectively; or
HCDR1 having the amino acid sequence as shown in SEQ ID NO: 05, HCDR2 having the amino acid sequence as shown in SEQ ID NO: 15, and HCDR3 having the amino acid sequence as shown in SEQ ID NO: 26 respectively; or
HCDR1 having the amino acid sequence as shown in SEQ ID NO: 06, HCDR2 having the amino acid sequence as shown in SEQ ID NO: 16, and HCDR3 having the amino acid sequence as shown in SEQ ID NO: 27 respectively; or
HCDR1 having the amino acid sequence as shown in SEQ ID NO: 07, HCDR2 having the amino acid sequence as shown in SEQ ID NO: 17, and HCDR3 having the amino acid sequence as shown in SEQ ID NO: 28 respectively; or
HCDR1 having the amino acid sequence as shown in SEQ ID NO: 01, HCDR2 having the amino acid sequence as shown in SEQ ID NO: 18, and HCDR3 having the amino acid sequence as shown in SEQ ID NO: 22 respectively; or
HCDR1 having the amino acid sequence as shown in SEQ ID NO: 08, HCDR2 having the amino acid sequence as shown in SEQ ID NO: 19, and HCDR3 having the amino acid sequence as shown in SEQ ID NO: 29 respectively; or
HCDR1 having the amino acid sequence as shown in SEQ ID NO: 09, HCDR2 having the amino acid sequence as shown in SEQ ID NO: 20, and HCDR3 having the amino acid sequence as shown in SEQ ID NO: 30 respectively; or
HCDR1 having the amino acid sequence as shown in SEQ ID NO: 10, HCDR2 having the amino acid sequence as shown in SEQ ID NO: 21, and HCDR3 having the amino acid sequence as shown in SEQ ID NO: 31 respectively.

In some embodiments, the light chain variable region sequence comprises:
LCDR1 having the amino acid sequence as shown in SEQ ID NO: 32, LCDR2 having the amino acid sequence as shown in SEQ ID NO: 43, and LCDR3 having the amino acid sequence as shown in SEQ ID NO:48, respectively; or
LCDR1 having the amino acid sequence as shown in SEQ ID NO: 33, LCDR2 having the amino acid sequence as shown in SEQ ID NO: 44, and LCDR3 having the amino acid sequence as shown in SEQ ID NO: 49, respectively; or
LCDR1 having the amino acid sequence as shown in SEQ ID NO: 34, LCDR2 having the amino acid sequence as shown in SEQ ID NO: 43, and LCDR3 having the amino acid sequence as shown in SEQ ID NO: 50, respectively; or
LCDR1 having the amino acid sequence as shown in SEQ ID NO: 35, LCDR2 having the amino acid sequence as shown in SEQ ID NO: 43, and LCDR3 having the amino acid sequence as shown in SEQ ID NO: 49, respectively; or
LCDR1 having the amino acid sequence as shown in SEQ ID NO: 36, LCDR2 having the amino acid sequence as shown in SEQ ID NO: 43, and LCDR3 having the amino acid sequence as shown in SEQ ID NO: 51, respectively; or
LCDR1 having the amino acid sequence as shown in SEQ ID NO: 37, LCDR2 having the amino acid sequence as shown in SEQ ID NO: 43, and LCDR3 having the amino acid sequence as shown in SEQ ID NO: 52, respectively; or
LCDR1 having the amino acid sequence as shown in SEQ ID NO: 38, LCDR2 having the amino acid sequence as shown in SEQ ID NO: 45, and LCDR3 having the amino acid sequence as shown in SEQ ID NO: 53, respectively; or
LCDR1 having the amino acid sequence as shown in SEQ ID NO: 39, LCDR2 having the amino acid sequence as shown in SEQ ID NO: 43, and LCDR3 having the amino acid sequence as shown in SEQ ID NO: 48, respectively; or
LCDR1 having the amino acid sequence as shown in SEQ ID NO: 40, LCDR2 having the amino acid sequence as shown in SEQ ID NO: 46, and LCDR3 having the amino acid sequence as shown in SEQ ID NO: 51, respectively; or
LCDR1 having the amino acid sequence as shown in SEQ ID NO: 41, LCDR2 having the amino acid sequence as shown in SEQ ID NO: 43, and LCDR3 having the amino acid sequence as shown in SEQ ID NO: 52, respectively; or
LCDR1 having the amino acid sequence as shown in SEQ ID NO: 42, LCDR2 having the amino acid sequence as shown in SEQ ID NO: 47, and LCDR3 having the amino acid sequence as shown in SEQ ID NO: 54, respectively.

In a particular embodiment, the antibody or antigen-binding fragment thereof comprises:
a) a heavy chain variable region comprises HCDR1, HCDR2 and HCDR3 having the amino acid sequences as shown in SEQ ID NO: 01, SEQ ID NO: 11 and SEQ ID NO: 22, respectively; and a light chain variable region comprises LCDR1, LCDR2 and LCDR3 having the amino acid sequences as shown in SEQ ID NO: 32, SEQ ID NO: 43 and SEQ ID NO: 48, respectively; or
b) a heavy chain variable region comprises HCDR1, HCDR2 and HCDR3 having the amino acid sequences as shown in SEQ ID NO: 02, SEQ ID NO: 12 and SEQ ID NO: 23, respectively; and a light chain variable region sequence comprises LCDR1, LCDR2 and LCDR3 having the amino acid sequences as shown in SEQ ID NO: 33, SEQ ID NO: 44 and SEQ ID NO: 49, respectively; or
c) a heavy chain variable region comprises HCDR1, HCDR2 and HCDR3 having the amino acid sequences as shown in SEQ ID NO: 03, SEQ ID NO: 13 and SEQ ID NO: 24, respectively; and a light chain variable region comprises LCDR1, LCDR2 and LCDR3 having the amino acid sequences as shown in SEQ ID NO: 34, SEQ ID NO: 43 and SEQ ID NO: 50, respectively; or
d) a heavy chain variable region comprises HCDR1, HCDR2 and HCDR3 having the amino acid sequences as shown in SEQ ID NO: 04, SEQ ID NO: 14 and SEQ ID NO: 25, respectively; and a light chain variable region comprises LCDR1, LCDR2 and LCDR3 having the amino acid sequences as shown in SEQ ID NO: 35, SEQ ID NO: 43 and SEQ ID NO: 49, respectively; or
e) a heavy chain variable region comprises HCDR1, HCDR2 and HCDR3 having the amino acid sequences as shown in SEQ ID NO: 05, SEQ ID NO: 15 and SEQ ID NO: 26, respectively; and a light chain variable region comprises LCDR1, LCDR2 and LCDR3 having the amino acid sequences as shown in SEQ ID NO: 36, SEQ ID NO: 43 and SEQ ID NO: 51, respectively; or
f) a heavy chain variable region comprises HCDR1, HCDR2 and HCDR3 having the amino acid sequences as shown in SEQ ID NO: 06, SEQ ID NO: 16 and SEQ ID NO: 27, respectively; and a light chain variable region comprises LCDR1, LCDR2 and LCDR3 having the amino acid sequences as shown in SEQ ID NO: 37, SEQ ID NO: 43 and SEQ ID NO: 52, respectively; or
g) a heavy chain variable region comprises HCDR1, HCDR2 and HCDR3 having the amino acid sequences as shown in SEQ ID NO: 07, SEQ ID NO: 17 and SEQ ID NO: 28, respectively; and a light chain variable region comprises LCDR1, LCDR2 and LCDR3 having the amino acid sequences as shown in SEQ ID NO: 38, SEQ ID NO: 45 and SEQ ID NO: 53, respectively; or
h) a heavy chain variable region comprises HCDR1, HCDR2 and HCDR3 having the amino acid sequences as shown in SEQ ID NO: 01, SEQ ID NO: 18 and SEQ ID NO: 22, respectively; and a light chain variable region comprises LCDR1, LCDR2 and LCDR3 having the amino acid sequences as shown in SEQ ID NO: 39, SEQ ID NO: 43 and SEQ ID NO: 48, respectively; or
i) a heavy chain variable region comprises HCDR1, HCDR2 and HCDR3 having the amino acid sequences as shown in SEQ ID NO: 08, SEQ ID NO: 19 and SEQ ID NO: 29, respectively; and a light chain variable region comprises LCDR1, LCDR2 and LCDR3 having the amino acid sequences as shown in SEQ ID NO: 40, SEQ ID NO: 46 and SEQ ID NO: 51, respectively; or
j) a heavy chain variable region comprises HCDR1, HCDR2 and HCDR3 having the amino acid sequences as shown in SEQ ID NO: 09, SEQ ID NO: 20 and SEQ ID NO: 30, respectively; and a light chain variable region comprises LCDR1, LCDR2 and LCDR3 having the amino acid sequences as shown in SEQ ID NO: 41, SEQ ID NO: 43 and SEQ ID NO: 52, respectively; or
k) a heavy chain variable region comprises HCDR1, HCDR2 and HCDR3 having the amino acid sequences as shown in SEQ ID NO: 10, SEQ ID NO: 21 and SEQ ID NO: 31, respectively; and a light chain variable region comprises LCDR1, LCDR2 and LCDR3 having the amino acid sequences as shown in SEQ ID NO: 42, SEQ ID NO: 47 and SEQ ID NO: 54, respectively.

In a particular embodiment, the antibody or antigen-binding fragment thereof is a monoclonal antibody or antigen-binding fragment thereof, a polyclonal antibody or antigen-binding fragment thereof, a bispecific antibody or antigen-binding fragment thereof, a murine antibody or antigen-binding fragment thereof, a chimeric antibody or antigen-binding fragment thereof, a humanized antibody or antigen-binding fragment thereof, a recombinant antibody or antigen-binding fragment thereof, a human antibody or antigen-binding fragment thereof.

In some embodiments, the antibody or antigen-binding fragment thereof comprises: a heavy chain variable region comprising an amino acid sequence selected from: SEQ ID NOs: 55-65, or sequence having at least 80%, 85%, 90%, 95% or 99% sequence identity therewith; and/or a light chain variable region comprising an amino acid sequence selected from: SEQ ID NOs: 66-76, or sequence having at least 80%, 85%, 90%, 95% or 99% sequence identity therewith.

In some embodiments, the antibody or antigen-binding fragment thereof comprises:
a) the heavy chain variable region having the amino acid sequence as shown in SEQ ID NO: 55, or having the amino acid sequence of at least 80%, 85%, 90%, 95% or 99% sequence identity therewith; and/or the light chain variable region having the amino acid sequence as shown in SEQ ID NO: 66, or having the amino acid sequence of at least 80%, 85%, 90%, 95% or 99% sequence identity therewith; or
b) the heavy chain variable region having the amino acid sequence as shown in SEQ ID NO: 56, or having the amino acid sequence of at least 80%, 85%, 90%, 95% or 99% sequence identity therewith; and/or the light chain variable region having the amino acid sequence as shown in SEQ ID NO: 67, or having the amino acid sequence of at least 80%, 85%, 90%, 95% or 99% sequence identity therewith; or
c) the heavy chain variable region having the amino acid sequence as shown in SEQ ID NO: 57, or having the amino acid sequence of at least 80%, 85%, 90%, 95% or 99% sequence identity therewith; and/or the light chain variable region having the amino acid sequence as shown in SEQ ID NO: 68, or having the amino acid sequence of at least 80%, 85%, 90%, 95% or 99% sequence identity therewith; or
d) the heavy chain variable region having the amino acid sequence as shown in SEQ ID NO: 58, or having the amino acid sequence of at least 80%, 85%, 90%, 95% or 99% sequence identity therewith; and/or the light chain variable region having the amino acid sequence as shown in SEQ ID NO: 69, or having the amino acid sequence of at least 80%, 85%, 90%, 95% or 99% sequence identity therewith; or
e) the heavy chain variable region having the amino acid sequence as shown in SEQ ID NO: 59, or having the amino acid sequence of at least 80%, 85%, 90%, 95% or 99% sequence identity therewith; and/or the light chain variable region having the amino acid sequence as shown in SEQ ID NO: 70, or having the amino acid sequence of at least 80%, 85%, 90%, 95% or 99% sequence identity therewith; or
f) the heavy chain variable region having the amino acid sequence as shown in SEQ ID NO: 60, or having the amino acid sequence of at least 80%, 85%, 90%, 95% or 99% sequence identity therewith; and/or the light chain variable region having the amino acid sequence as shown in SEQ ID NO: 71, or having the amino acid sequence of at least 80%, 85%, 90%, 95% or 99% sequence identity therewith; or
g) the heavy chain variable region having the amino acid sequence as shown in SEQ ID NO: 61, or having the amino acid sequence of at least 80%, 85%, 90%, 95% or 99% sequence identity therewith; and/or the light chain variable region having the amino acid sequence as shown in SEQ ID NO: 72, or having the amino acid sequence of at least 80%, 85%, 90%, 95% or 99% sequence identity therewith; or
h) the heavy chain variable region having the amino acid sequence as shown in SEQ ID NO: 62, or having the amino acid sequence of at least 80%, 85%, 90%, 95% or 99% sequence identity therewith; and/or the light chain variable region having the amino acid sequence as shown in SEQ ID NO: 73, or having the amino acid sequence of at least 80%, 85%, 90%, 95% or 99% sequence identity therewith; or
i) the heavy chain variable region having the amino acid sequence as shown in SEQ ID NO: 63, or having the amino acid sequence of at least 80%, 85%, 90%, 95% or 99% sequence identity therewith; and/or the light chain variable region having the amino acid sequence as shown in SEQ ID NO: 74, or having the amino acid sequence of at least 80%, 85%, 90%, 95% or 99% sequence identity therewith; or
j) the heavy chain variable region having the amino acid sequence as shown in SEQ ID NO: 64, or having the amino acid sequence of at least 80%, 85%, 90%, 95% or 99% sequence identity therewith; and/or the light chain variable region having the amino acid sequence as shown in SEQ ID NO: 75, or having the amino acid sequence of at least 80%, 85%, 90%, 95% or 99% sequence identity therewith; or
k) the heavy chain variable region having the amino acid sequence as shown in SEQ ID NO: 65, or having the amino acid sequence of at least 80%, 85%, 90%, 95% or 99% sequence identity therewith; and/or the light chain variable region having the amino acid sequence as shown in SEQ ID NO: 76, or having the amino acid sequence of at least 80%, 85%, 90%, 95% or 99% sequence identity therewith.

In a particular embodiment, the antibody or antigen-binding fragment thereof comprises: a) the heavy chain variable region having the amino acid sequence as shown in SEQ ID NO: 55; and/or the light chain variable region having the amino acid sequence as shown in SEQ ID NO: 66; or b) the heavy chain variable region having the amino acid sequence as shown in SEQ ID NO: 56; and/or the light chain variable region having the amino acid sequence as shown in SEQ ID NO: 67; or c) the heavy chain variable region having the amino acid sequence as shown in SEQ ID NO: 57; and/or the light chain variable region having the amino acid sequence as shown in SEQ ID NO: 68; or d) the heavy chain variable region having the amino acid sequence as shown in SEQ ID NO: 58; and/or the light chain variable region having the amino acid sequence as shown in SEQ ID NO: 69; or e) the heavy chain variable region having the amino acid sequence as shown in SEQ ID NO: 59; and/or the light chain variable region having the amino acid sequence as shown in SEQ ID NO: 70; or f) the heavy chain variable region having the amino acid sequence as shown in SEQ ID NO: 60; and/or the light chain variable region having the amino acid sequence as shown in SEQ ID NO: 71; or g) the heavy chain variable region having the amino acid sequence as shown in SEQ ID NO: 61; and/or the light chain variable region having the amino acid sequence as shown in SEQ ID NO: 72; or h) the heavy chain variable region having the amino acid sequence as shown in SEQ ID NO: 62; and/or the light chain variable region having the amino acid sequence as shown in SEQ ID NO: 73; or i) the heavy chain variable region having the amino acid sequence as shown in SEQ ID NO: 63; and/or the light chain variable region having the amino acid sequence as shown in SEQ ID NO: 74; or j) the heavy chain variable region having the amino acid sequence as shown in SEQ ID NO: 64; and/or the light chain variable region having the amino acid sequence as shown in SEQ ID NO: 75; or k) the heavy chain variable region having the amino acid sequence as shown in SEQ ID NO: 65; and/or the light chain variable region having the amino acid sequence as shown in SEQ ID NO: 76.

In some embodiments, the antibody or antigen-binding fragment thereof comprises: a) the heavy chain variable region having the amino acid sequence as shown in SEQ ID NO: 55; and the light chain variable region having the amino acid sequence as shown in SEQ ID NO: 66; or b) the heavy chain variable region having the amino acid sequence as shown in SEQ ID NO: 56; and the light chain variable region having the amino acid sequence as shown in SEQ ID NO: 67; or c) the heavy chain variable region having the amino acid sequence as shown in SEQ ID NO: 57; and the light chain variable region having the amino acid sequence as shown in SEQ ID NO: 68; or d) the heavy chain variable region having the amino acid sequence as shown in SEQ ID NO: 58; and the light chain variable region having the amino acid sequence as shown in SEQ ID NO: 69; or e) the heavy chain variable region having the amino acid sequence as shown in SEQ ID NO: 59; and the light chain variable region having the amino acid sequence as shown in SEQ ID NO: 70; or f) the heavy chain variable region having the amino acid sequence as shown in SEQ ID NO: 60; and the light chain variable region having the amino acid sequence as shown in SEQ ID NO: 71; or g) the heavy chain variable region having the amino acid sequence as shown in SEQ ID NO: 61; and the light chain variable region having the amino acid sequence as shown in SEQ ID NO: 72; or h) the heavy chain variable region having the amino acid sequence as shown in SEQ ID NO: 62; and the light chain variable region having the amino acid sequence as shown in SEQ ID NO: 73; or i) the heavy chain variable region having the amino acid sequence as shown in SEQ ID NO: 63; and the light chain variable region having the amino acid sequence as shown in SEQ ID NO: 74; or j) the heavy chain variable region having the amino acid sequence as shown in SEQ ID NO: 64; and the light chain variable region having the amino acid sequence as shown in SEQ ID NO: 75; or k) the heavy chain variable region having the amino acid sequence as shown in SEQ ID NO: 65; and the light chain variable region having the amino acid sequence as shown in SEQ ID NO: 76.

In some embodiments, the antibody or antigen-binding fragment thereof further comprises human antibody constant regions; preferably, the heavy chain constant region of the human antibody constant regions is selected from the group consisting of: constant regions of human IgG1, IgG2, IgG3 and IgG4 and conventional variants thereof, and the light chain constant region of the human antibody constant regions is selected from the group consisting of: κ and λ chain constant regions of human antibody and conventional variants thereof; more preferably, the full-length antibody comprises a human antibody heavy chain constant region of SEQ ID NO: 99 or having the amino acid sequence of at least 80%, 85%, 90%, 95% or 99% sequence identity therewith, a human light chain constant region of SEQ ID NO: 100 or having the amino acid sequence of at least 80%, 85%, 90%, 95% or 99% sequence identity therewith; further preferably, the full-length antibody comprises a human antibody heavy chain constant region of SEQ ID NO: 99 and a human light chain constant region of SEQ ID NO: 100.

In a particular embodiment, the antibody or antigen-binding fragment thereof comprising:
a) a heavy chain having the amino acid sequence as shown in SEQ ID NO: 77 or having the amino acid sequence of at least 80%, 85%, 90%, 95% or 99% sequence identity therewith; and a light chain having the amino acid sequence as shown in SEQ ID NO: 78 or having the amino acid sequence of at least 80%, 85%, 90%, 95% or 99% sequence identity therewith; or
b) a heavy chain having the amino acid sequence as shown in SEQ ID NO: 79 or having the amino acid sequence of at least 80%, 85%, 90%, 95% or 99% sequence identity therewith; and a light chain having the amino acid sequence as shown in SEQ ID NO: 80 or having the amino acid sequence of at least 80%, 85%, 90%, 95% or 99% sequence identity therewith; or
c) a heavy chain having the amino acid sequence as shown in SEQ ID NO: 81 or having the amino acid sequence of at least 80%, 85%, 90%, 95% or 99% sequence identity therewith; and a light chain having the amino acid sequence as shown in SEQ ID NO: 82 or having the amino acid sequence of at least 80%, 85%, 90%, 95% or 99% sequence identity therewith; or
d) a heavy chain having the amino acid sequence as shown in SEQ ID NO: 83 or having the amino acid sequence of at least 80%, 85%, 90%, 95% or 99% sequence identity therewith; and a light chain having the amino acid sequence as shown in SEQ ID NO: 84 or having the amino acid sequence of at least 80%, 85%, 90%, 95% or 99% sequence identity therewith; or
e) a heavy chain having the amino acid sequence as shown in SEQ ID NO: 85 or having the amino acid sequence of at least 80%, 85%, 90%, 95% or 99% sequence identity therewith; and a light chain having the amino acid sequence as shown in SEQ ID NO: 86 or having the amino acid sequence of at least 80%, 85%, 90%, 95% or 99% sequence identity therewith; or
f) a heavy chain having the amino acid sequence as shown in SEQ ID NO: 87 or having the amino acid sequence of at least 80%, 85%, 90%, 95% or 99% sequence identity therewith; and a light chain having the amino acid sequence as shown in SEQ ID NO: 88 or having the amino acid sequence of at least 80%, 85%, 90%, 95% or 99% sequence identity therewith; or
g) a heavy chain having the amino acid sequence as shown in SEQ ID NO: 89 or having the amino acid sequence of at least 80%, 85%, 90%, 95% or 99% sequence identity therewith; and a light chain having the amino acid sequence as shown in SEQ ID NO: 90 or having the amino acid sequence of at least 80%, 85%, 90%, 95% or 99% sequence identity therewith; or
h) a heavy chain having the amino acid sequence as shown in SEQ ID NO: 91 or having the amino acid sequence of at least 80%, 85%, 90%, 95% or 99% sequence identity therewith; and a light chain having the amino acid sequence as shown in SEQ ID NO: 92 or having the amino acid sequence of at least 80%, 85%, 90%, 95% or 99% sequence identity therewith; or
i) a heavy chain having the amino acid sequence as shown in SEQ ID NO: 93 or having the amino acid sequence of at least 80%, 85%, 90%, 95% or 99% sequence identity therewith; and a light chain having the amino acid sequence as shown in SEQ ID NO: 94 or having the amino acid sequence of at least 80%, 85%, 90%, 95% or 99% sequence identity therewith; or
j) a heavy chain having the amino acid sequence as shown in SEQ ID NO: 95 or having the amino acid sequence of at least 80%, 85%, 90%, 95% or 99% sequence identity therewith; and a light chain having the amino acid sequence as shown in SEQ ID NO: 96 or having the amino acid sequence of at least 80%, 85%, 90%, 95% or 99% sequence identity therewith; or
k) a heavy chain having the amino acid sequence as shown in SEQ ID NO: 97 or having the amino acid sequence of at least 80%, 85%, 90%, 95% or 99% sequence identity therewith; and a light chain having the amino acid sequence as shown in SEQ ID NO: 98 or having the amino acid sequence of at least 80%, 85%, 90%, 95% or 99% sequence identity therewith.

In a particular embodiment, the antibody or antigen-binding fragment thereof comprising: a) a heavy chain having the amino acid sequence as shown in SEQ ID NO:77 and a light chain having the amino acid sequence as shown in SEQ ID NO:78; or b) a heavy chain having the amino acid sequence as shown in SEQ ID NO:79 and a light chain having the amino acid sequence as shown in SEQ ID NO:80; or c) a heavy chain having the amino acid sequence as shown in SEQ ID NO:81 and a light chain having the amino acid sequence as shown in SEQ ID NO:82; or d) a heavy chain having the amino acid sequence as shown in SEQ ID NO:83 and a light chain having the amino acid sequence as shown in SEQ ID NO:84; or e) a heavy chain having the amino acid sequence as shown in SEQ ID NO:85 and a light chain having the amino acid sequence as shown in SEQ ID NO:86: or f) a heavy chain having the amino acid sequence as shown in SEQ ID NO:87 and a light chain having the amino acid sequence as shown in SEQ ID NO:88; or g) a heavy chain having the amino acid sequence as shown in SEQ ID NO:89 and a light chain having the amino acid sequence as shown in SEQ ID NO:90; or h) a heavy chain having the amino acid sequence as shown in SEQ ID NO:91 and a light chain having the amino acid sequence as shown in SEQ ID NO:92; or i) a heavy chain having the amino acid sequence as shown in SEQ ID NO:93 and a light chain having the amino acid sequence as shown in SEQ ID NO:94; or j) a heavy chain having the amino acid sequence as shown in SEQ ID NO:95 and a light chain having the amino acid sequence as shown in SEQ ID NO:96; or k) a heavy chain having the amino acid sequence as shown in SEQ ID NO:97 and a light chain having the amino acid sequence as shown in SEQ ID NO:98.

In a particular embodiment, the antigen-binding fragment is selected from the group consisting of Fab, Fab′, F(ab′)2, variable fragment (Fv), single chain variable fragment (scFv), dimerized domain V (diabody), disulfide stabilized Fv (dsFv) and CDR-containing peptides.

In some embodiments, the antibody or antigen-binding fragment comprises: a heavy chain variable region comprising HCDR1, HCDR2 and HCDR3 regions and a light chain variable region comprising LCDR1, LCDR2 and LCDR3 regions, wherein:
a heavy chain variable region comprises HCDR1, HCDR2 and HCDR3 having the amino acid sequences as shown in SEQ ID NO: 05, SEQ ID NO: 15 and SEQ ID NO: 26, respectively; and a light chain variable region comprises LCDR1, LCDR2 and LCDR3 having the amino acid sequences as shown in SEQ ID NO: 36, SEQ ID NO: 43 and SEQ ID NO: 51, respectively; or
a heavy chain variable region comprises HCDR1, HCDR2 and HCDR3 having the amino acid sequences as shown in SEQ ID NO: 07, SEQ ID NO:17 and SEQ ID NO: 28, respectively; and a light chain variable region comprises LCDR1, LCDR2 and LCDR3 having the amino acid sequences as shown in SEQ ID NO: 38, SEQ ID NO: 45 and SEQ ID NO: 53, respectively;
or a heavy chain variable region comprises HCDR1, HCDR2 and HCDR3 having the amino acid sequences as shown in SEQ ID NO: 10, SEQ ID NO:21 and SEQ ID NO: 31, respectively; and a light chain variable region comprises LCDR1, LCDR2 and LCDR3 having the amino acid sequences as shown in SEQ ID NO: 42, SEQ ID NO: 47 and SEQ ID NO: 54, respectively, and
wherein said antibody or antigen-binding fragment specifically binds to both human and cynomolgus CDH17; preferably, wherein said antibody or antigen-binding fragment binds to both human and cynomolgus CDH17 with a K_{D}≤1000 nM; more preferably, wherein said antibody or antigen-binding fragment binds to both human and cynomolgus CDH17 with a K_{D}≤500 nM, 400 nM, 300 nM, 200 nM, 100 nM, 90 nM, 80 nM, 70 nM, 60 nM, 50 nM, 40 nM, 30 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM or 3 nM; further preferably, wherein said antibody or antigen-binding fragment has the following properties: a) binds to human CDH17 with a K_{D} of 15 nM or less; b) binds to cynomolgus CDH17 with a K_{D} of 15 nM or less.

In some embodiments, the antibody or antigen-binding fragment comprises: a heavy chain variable region comprising HCDR1, HCDR2 and HCDR3 regions and a light chain variable region comprising LCDR1, LCDR2 and LCDR3 regions, wherein:
a heavy chain variable region comprises HCDR1, HCDR2 and HCDR3 having the amino acid sequences as shown in SEQ ID NO: 05, SEQ ID NO: 15 and SEQ ID NO: 26, respectively; and a light chain variable region comprises LCDR1, LCDR2 and LCDR3 having the amino acid sequences as shown in SEQ ID NO: 36, SEQ ID NO: 43 and SEQ ID NO: 51, respectively;
or a heavy chain variable region comprises HCDR1, HCDR2 and HCDR3 having the amino acid sequences as shown in SEQ ID NO: 07, SEQ ID NO: 17 and SEQ ID NO: 28, respectively; and a light chain variable region comprises LCDR1, LCDR2 and LCDR3 having the amino acid sequences as shown in SEQ ID NO: 38, SEQ ID NO: 45 and SEQ ID NO: 53, respectively;
or a heavy chain variable region comprises HCDR1, HCDR2 and HCDR3 having the amino acid sequences as shown in SEQ ID NO: 10, SEQ ID NO:21 and SEQ ID NO: 31, respectively; and a light chain variable region comprises LCDR1, LCDR2 and LCDR3 having the amino acid sequences as shown in SEQ ID NO: 42, SEQ ID NO: 47 and SEQ ID NO: 54, respectively, and wherein said antibody or antigen-binding fragment has the following properties: a) binds to human CDH17 with a K_{D} of 3 nM or less; b) binds to cynomolgus CDH17 with a K_{D} of 3 nM or less; preferably, wherein said antibody or antigen-binding fragment has the following properties: a) binds to human CDH17 with a K_{D} of 2 nM or less; b) binds to cynomolgus CDH17 with a K_{D} of 3 nM or less.

In some embodiments, the antibody or antigen-binding fragment comprises: a heavy chain variable region comprising HCDR1, HCDR2 and HCDR3 regions and a light chain variable region comprising LCDR1, LCDR2 and LCDR3 regions, wherein:
a heavy chain variable region comprises HCDR1, HCDR2 and HCDR3 having the amino acid sequences as shown in SEQ ID NO: 10, SEQ ID NO:21 and SEQ ID NO: 31, respectively; and a light chain variable region comprises LCDR1, LCDR2 and LCDR3 having the amino acid sequences as shown in SEQ ID NO: 42, SEQ ID NO: 47 and SEQ ID NO: 54, respectively, and wherein said antibody or antigen-binding fragment specifically binds to both human and cynomolgus CDH17; preferably, wherein said antibody or antigen-binding fragment binds to both human and cynomolgus CDH17 with a K_{D}≤1000 nM; more preferably, wherein said antibody or antigen-binding fragment binds to both human and cynomolgus CDH17 with a K_{D}≤500 nM, 400 nM, 300 nM, 200 nM, 100 nM, 90 nM, 80 nM, 70 nM, 60 nM, 50 nM, 40 nM, 30 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM or 3 nM; further preferably, wherein said antibody or antigen-binding fragment has the following properties: a) binds to human CDH17 with a K_{D} of 15 nM or less; b) binds to cynomolgus CDH17 with a K_{D} of 15 nM or less.

In some embodiments, the antibody or antigen-binding fragment comprises: a heavy chain variable region comprising HCDR1, HCDR2 and HCDR3 regions and a light chain variable region comprising LCDR1, LCDR2 and LCDR3 regions, wherein:
a heavy chain variable region comprises HCDR1, HCDR2 and HCDR3 having the amino acid sequences as shown in SEQ ID NO: 10, SEQ ID NO:21 and SEQ ID NO: 31, respectively; and a light chain variable region comprises LCDR1, LCDR2 and LCDR3 having the amino acid sequences as shown in SEQ ID NO: 42, SEQ ID NO: 47 and SEQ ID NO: 54, respectively, and wherein said antibody or antigen-binding fragment has the following properties: a) binds to human CDH17 with a K_{D} of 3 nM or less; b) binds to cynomolgus CDH17 with a K_{D} of 3 nM or less; preferably, wherein said antibody or antigen-binding fragment has the following properties: a) binds to human CDH17 with a K_{D} of 2 nM or less; b) binds to cynomolgus CDH17 with a K_{D} of 3 nM or less.

In some embodiments, the present disclosure provides an isolated anti-CDH17 antibody or antigen-binding fragment thereof wherein the antibody or antigen-binding fragment competes with above mentioned antibody or antigen binding fragment for binding to CDH17.

In some embodiments, the antibody or antigen-binding fragment comprises: a heavy chain variable region comprising HCDR1, HCDR2 and HCDR3 regions and a light chain variable region comprising LCDR1, LCDR2 and LCDR3 regions, wherein: a heavy chain variable region comprises HCDR1, HCDR2 and HCDR3 having the amino acid sequences as shown in SEQ ID NO: 05, SEQ ID NO: 15 and SEQ ID NO: 26, respectively; and a light chain variable region comprises LCDR1, LCDR2 and LCDR3 having the amino acid sequences as shown in SEQ ID NO: 36, SEQ ID NO: 43 and SEQ ID NO: 51, respectively; or a heavy chain variable region comprises HCDR1, HCDR2 and HCDR3 having the amino acid sequences as shown in SEQ ID NO: 07, SEQ ID NO: 17 and SEQ ID NO: 28, respectively; and a light chain variable region comprises LCDR1, LCDR2 and LCDR3 having the amino acid sequences as shown in SEQ ID NO: 38, SEQ ID NO: 45 and SEQ ID NO: 53, respectively; or a heavy chain variable region comprises HCDR1, HCDR2 and HCDR3 having the amino acid sequences as shown in SEQ ID NO: 10, SEQ ID NO:21 and SEQ ID NO: 31, respectively; and a light chain variable region comprises LCDR1, LCDR2 and LCDR3 having the amino acid sequences as shown in SEQ ID NO: 42, SEQ ID NO: 47 and SEQ ID NO: 54, respectively, and wherein said antibody or antigen-binding fragment competes with antibody 20B4, 14B12 or 10E11 for binding to human CDH17.

In some embodiments, the antibody or antigen-binding fragment comprises: a heavy chain variable region comprising HCDR1, HCDR2 and HCDR3 regions and a light chain variable region comprising LCDR1, LCDR2 and LCDR3 regions, wherein: a heavy chain variable region comprises HCDR1, HCDR2 and HCDR3 having the amino acid sequences as shown in SEQ ID NO: 10, SEQ ID NO:21 and SEQ ID NO: 31, respectively; and a light chain variable region comprises LCDR1, LCDR2 and LCDR3 having the amino acid sequences as shown in SEQ ID NO: 42, SEQ ID NO: 47 and SEQ ID NO: 54, respectively, and wherein said antibody or antigen-binding fragment competes with antibody 20B4 for binding to human CDH17.

In some embodiments, the present disclosure provides an isolated nucleic acid molecule encoding any above-mentioned antibody or the antigen-binding fragment thereof.

In one aspect, the present disclosure also provides a recombinant vector comprising the above-mentioned isolated nucleic acid molecule.

In one aspect, the present disclosure also provides a host cell comprising the above-mentioned recombinant vector, wherein the host cell is selected from the group consisting of a prokaryotic cell and a eukaryotic cell, preferably a eukaryotic cell, more preferably a mammalian cell.

In one aspect, the present disclosure also provides a therapeutic conjugate comprising the above-mentioned anti-CDH17 antibody or the antigen-binding fragment thereof, wherein the antibody or the antigen-binding fragment thereof is conjugated with one or more therapeutic agents; preferably, the therapeutic agent is a cytotoxic agent.

In one aspect, the present disclosure also provides a diagnostic conjugate comprising the above-mentioned anti-CDH17 antibody or the antigen-binding fragment thereof, wherein the antibody or the antigen-binding fragment thereof is conjugated with one or more diagnostic agents; preferably, the diagnostic agent could be, but not limited to, a detectable label, enzyme, metal-containing substance, fluorescent substance, luminescent substance, colored substance, quantum dot.

In one aspect, the present disclosure also provides a bispecific antibody comprising a first binding domain that specifically binds to CDH17, and a second binding domain that specifically binds to a second target protein, wherein the first binding domain is the above-mentioned anti-CDH17 antibody or the antigen-binding fragment thereof.

In one aspect, the present disclosure also provides a method for producing the above-mentioned anti-CDH17 antibody or the antigen-binding fragment thereof, wherein the method comprises culturing the host cell in a medium to produce and accumulate the antibody or the antigen-binding fragment thereof, and harvesting the antibody or the antigen-binding fragment thereof from the culture.

In one aspect, the present disclosure also provides a method for immunologically detecting or measuring CDH17, wherein the method comprises detecting or measuring the CDH17 by contacting a sample with the above-mentioned anti-CDH17 antibody or antigen-binding fragment thereof or diagnostic conjugate.

In one aspect, the present disclosure also provides a method for diagnosing a disease related to a human CDH17 positive cell, wherein the method comprises detecting or measuring the CDH17 positive cell by contacting a sample with the above-mentioned anti-CDH17 antibody or antigen-binding fragment thereof or the diagnostic conjugate.

In one aspect, the present disclosure also provides a pharmaceutical composition, which comprises a therapeutically effective amount or prophylactically effective amount of the above-mentioned anti-CDH17 antibody or the antigen-binding fragment thereof or the bispecific antibody or the therapeutic conjugate, and one or more pharmaceutically acceptable carriers, diluents, or excipients.

In one aspect, the present disclosure also provides a method of treatment or prevention of a CDH17-mediated disease or disorder, comprises a step of administering a therapeutically effective amount or prophylactically effective amount of the above-mentioned anti-CDH17 antibody or antigen-binding fragment, or the bispecific antibody, or the therapeutic conjugate or the pharmaceutical composition, to a subject in need of treatment or prevention of the disease or disorder.

In some embodiments, the disease is cancer; preferably the cancer is colorectal cancer, gastric cancer, pancreatic cancer, liver cancer, gallbladder cancer, ovarian cancer or cholangiocarcinoma.

In another aspect, the present disclosure provides a kit comprising means to implement the method of the present disclosure. In a particular embodiment, the present disclosure provides a kit comprising at least one container comprising the above-mentioned anti-CDH17 antibody or antigen-binding fragment, the therapeutic conjugate or the pharmaceutical composition. The kit can further include and instructions for preparing, administering and/or delivering the above-mentioned anti-CDH17 antibody or antigen-binding fragment, the therapeutic conjugate or the pharmaceutical composition.

Advantages of the present disclosure: The antibody or the antigen-binding fragment thereof can specifically recognize/bind to both human and cynomolgus CDH17 with desirable affinity. Also the antibody or the antigen-binding fragment thereof has sufficient internalization in a CDH17 expressing human cell, and can effectively bind to CDH17 and mediate the killing of CDH17 expressing cells.

### BRIEF DESCRIPTION OF THE FIGURES

FIG. 1. In vitro binding characterization of hybridoma clones to AsPC1 (A), GP2d (B), SW480 (C) cells.

FIG. 2. Cell internalization activity of selected hybridoma clones were characterized using indirect killing assay.

FIG. 3. In vitro binding characterization of anti-CDH17 recombinant antibodies to AsPC1 (A) cells and SW480 cells (B) was determined by flow cytometry analysis, with HBMAB81 served as positive control and B12 as isotype control antibody (C).

FIG. 4. Dose-response curves of anti-CDH17 recombinant antibody internalization by indirect-killing assay.

FIG. 5. Binding activities of HBMAB81 to cross-species CDH17, homologs CDH6 or CDH16 by Octet characterization.

FIG. 6. Binding activities of anti-CDH17 clone 10E11 to cross-species CDH17, homologs CDH6 or CDH16 by Octet characterization.

FIG. 7. Binding activities of anti-CDH17 clone 14B12 to cross-species CDH17, homologs CDH6 or CDH16 by Octet characterization.

FIG. 8. Binding activities of anti-CDH17 clone 20B4 to cross-species CDH17, homologs CDH6 or CDH16 by Octet characterization.

FIG. 9. Binding competition: (A) 20B4; (B) HBMAB81.

### DETAILED DESCRIPTION OF THE EMBODIMENTS

### Definitions of Terms

Before the present disclosure is detailed below, it is to be understood that the present disclosure is not limited to the particular methodologies, protocols and reagents described herein, as those may vary. It is also to be understood that the terminology used herein is for the purpose of describing the particular embodiments only, and is not intended to limit the scope of the invention, which will be limited only by the appended claims. Unless defined otherwise, all technical and scientific terms used herein have the same meaning as commonly understood by one of ordinary skill in the art to which the invention pertains.

For interpretation of the specification, the following definitions will be applied and wherever appropriate, terms used in the singular form may also include the plural counterpart and vice versa.

It is to be understood that the terminology used herein is for the purpose of describing specific embodiments only and is not intended to be limiting.

The terms used herein are for the purpose of describing specific embodiments only and are not intended to limit the present disclosure. Unless the context clearly indicates otherwise, the open-ended expressions “include” and “including” shall be interpreted as including structural components or method steps that are not mentioned, but it should be noted that the open-ended expression also covers the situation that only the components and method steps are mentioned (that is, it covers the situation that the closed-ended expression “consists of . . . ).

The terms “CDH17” and “CDH17 antigen” are used interchangeably herein, and include any variants, isoforms and species homologs of human CDH17 which are naturally expressed by cells or are expressed on cells transfected with the CDH17 gene.

“CDH17” as the target should be widely interpreted herein, aims to cover various forms of molecules of CDH17 at various stages in a mammalian (such as human), such as but not limited to molecules generated during the amplification, replication, transcription, splicing, translation, and modification of CDH17 gene (such as precursor CDH17, mature CDH17, membrane-expressed CDH17, CDH17 splicing variants, modified CDH17, or fragments thereof). This term also covers artificially prepared or in vitro expressed CDH17.

The term “antibody” as referred to herein includes whole antibodies and any antigen binding fragment (i. e., “antigen-binding portion”) or single chain thereof.

The term “antibody” refers to an immunoglobulin comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region (CH). Each light chain is comprised of a light chain variable region (abbreviated herein as VL) and a light chain constant region (CL). The VH and VL regions can be further subdivided into regions of hyper variability, termed complementarity determining regions (CDR), interspersed with regions that are more conserved, termed framework regions (FR). Both heavy chain and light chain have three complementary determination regions, which are called HCDR1, HCDR2, HCDR3 and LCDR1, LCDR2, LCDR3 respectively. Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen or epitope(s) thereof. The constant regions of the antibodies may mediate the binding of the immunoglobulin to host tissues or factors, including various cells of the immune system (e. g., effector cells) and the first component (C1q) of the classical complement system. Antibodies are assigned to classes based on the amino acid sequence of the constant region of their heavy chain. The five major classes or isotypes of antibodies are IgA, IgD, IgE, IgG, and IgM, and the corresponding heavy chains are p chain, 8 Chain, γ Chain, α Chain and ε Chain. The same type of Ig can be also classified into different subclasses, according to the amino acid composition of its hinge region and the number and position of heavy chain disulfide bonds, for example, IgG can be classified into IgG1, IgG2, IgG3 and IgG4. Generally, the heavy chain constant region of IgG antibody includes three constant domains, namely CH1, CH2 and CH3, and the light chain constant region includes one constant domain, namely CL. The light chain according to the constant region can also be classified into κ or λ Chain. Each of the five types of Ig can have κ Chain or λ Chain.

The term “antigen-binding fragment” of an antibody, as used herein, refers to one or more fragments of an antibody that retain the ability to specifically bind to an antigen or epitope(s) thereof (e. g., CDH17). It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the VL, VH, CL and CH1 domains; (ii) a F (ab′) 2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341: 544-546), which consists of a VH domain; (vi) an isolated complementarity determining region (CDR), and (vii) a combination of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e. g., Bird et al. (1988) Science 242: 423-426; and Huston et al. (1988) Proc. Natl. Acad. Sci. USA 85: 5879-5883). Such single chain antibodies are also intended to be encompassed within the term “antigen-binding portion” of an antibody. In some examples, the term “antigen-binding fragment” also include dimerized domain V (diabody) and disulfide stabilized Fv (dsFv).

The term “human antibody”, as used herein, is intended to include antibodies having variable and constant regions derived from human germline immunoglobulin sequences. The human antibodies of the present disclosure may include amino acid residues not encoded by human germline immunoglobulin sequences (e. g., mutations introduced by random or site-specific mutagenesis in vitro or by somatic mutation in vivo).

“Humanized antibody” include antibodies with variable and constant regions of a human immunoglobulin sequence, in which CDR sequences derived from another mammalian species (such as mice) germline have been grafted onto a human skeleton sequence.

“Chimeric antibody” usually refers to an antibody molecule formed by antibody domain fragments from different sources, as an example, an antibody that comprises a mouse derived variable region inserted into a human derived constant region.

The term “recombinant human antibody”, as used herein, includes all human antibodies that are prepared, expressed, created or isolated by recombinant means, such as (a) antibodies isolated from an animal (e. g., a mouse) that is transgenic or trans chromosomal for human immunoglobulin genes or a hybridoma prepared therefrom, (b) antibodies isolated from a host cell transformed to express the antibody, e. g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “CDR” refers to one of the six hypervariable regions within the variable domain of an antibody that primarily contributes to antigen binding. One of the most commonly used definitions for the six CDRs is provided by Kabat E. A. et al. (1991) Sequences of proteins of immunological interest. NIH Publication 91-3242. As used herein, the Kabat definition of CDR only applies to CDR1, CDR2 and CDR3 of the light chain variable domain (LCDR1, LCDR2, LCDR3 or L1, L2, L3), as well as CDR1, CDR2 and CDR3 of heavy chain variable domain (HCDR1, HCDR2, HCDR3 or H1, H2, H3). Also, CDRs can be defined according to other various numbering systems known in the art, for example, Chothia numbering system (Chothia & Lesk (1987) J. Mol. Biol. 196:901-917; Chothia et al. (1989) Nature 342:878-883), IMGT numbering system (Lefranc et al., Dev. Comparat. Immunol. 27:55-77, 2003), or the definition of Martin's related research (Martin A C R, Cheetham J C, Rees A R (1989) Modelling antibody hypervariable loops: A combined algorithm. Proc Natl Acad Sci USA 86:9268-9272), the correspondence between different numbering systems is well known to those skilled in the art (for example, refer to Lefranc et al., Dev. Comparat. Immunol. 27:55-77, 2003). Those skilled in the art can easily identify the CDRs defined by each numbering system.

“Conservative modification” or “conservative replacement or substitution” or “conventional variant” refers to substitutions of amino acids in a protein with other amino acid having similar characteristics (e.g., charge, side chain size, hydrophobicity/hydrophilicity, backbone conformation and rigidity, etc.), such that the substitutions can be frequently made without altering the biological activity of the protein. It will be appreciated by those skilled in the art that, in general, a single amino acid substitution in a non-essential region of polypeptide does not substantially alter biological activity (see, for example, Watson et al. (1987) Molecular Biology of the Gene, The Benjamin/Cummings Pub. Co., Page 224, (4th edition)). In addition, substitutions with structurally or functionally similar amino acids are unlikely to affect biological activity.

In some embodiments, an anti-CDH17 antibody or fragment thereof of the present disclosure encompasses an antibody or fragment thereof having a post-translational modification on a light chain variable region, a heavy chain variable region, a light chain or a heavy chain.

In certain embodiments, an antibody provided herein is modified to increase or decrease the extent of glycosylation. Addition or deletion of glycosylation sites of an antibody may be conveniently accomplished by altering the amino acid sequence such that one or more glycosylation sites is introduced or removed. In some applications, modifications that remove unwanted glycosylation sites may be useful, for example, modifications that remove fucose modules so as to enhance the antibody-dependent cellular cytotoxicity (ADCC) function (see Shield et al. (2002) JBC 277: 26733). In other applications, galactosylation modification may be performed to modify complement-dependent cytotoxicity (CDC).

In certain embodiments, one or more amino acid modifications may be introduced into the Fc region of an antibody provided herein, thereby generating an Fc region variant, so as to enhance the efficiency of the antibody, for example, in the treatment of cancer or cell proliferation disease. The Fc region variant may comprise human Fc region sequence (e.g., human IgG1, IgG2, IgG3 or IgG4 Fc region) comprising an amino acid modification (e.g. a substitution) at one or more amino acid positions.

The term “nucleic acid molecule” as used herein refers to a DNA molecule and a RNA molecule. The nucleic acid molecule may be single stranded or double stranded but is preferably a double stranded DNA. A nucleic acid is “effectively linked” when it is placed into functional relationship with another nucleic acid sequence. For example, a promoter or an enhancer is deemed to be effectively linked to a coding sequence, when the promoter or enhancer affects transcription of the coding sequence.

The preparation method of the nucleic acid is a conventional preparation method in the art. Preferably, it comprises the following steps: obtaining the nucleic acid molecule encoding the above-mentioned protein by gene cloning technology, or obtaining the nucleic acid molecule encoding the above-mentioned protein by the method of artificial full-length sequence synthesis.

Those skilled in the art know that the base sequence encoding the amino acid sequence of the protein can be replaced, deleted, changed, inserted or added appropriately to provide a polynucleotide homolog. The homolog of the polynucleotide of the present disclosure can be prepared by replacing, deleting or adding one or more bases of the gene encoding the protein sequence within the scope of maintaining the activity of the antibody.

The term “vector” refers to a nucleic acid molecule capable of transporting another nucleic acid to that it has been linked. In one embodiment, the vector is a “plasmid” that refers to a circular double stranded DNA loop into which additional DNA segment can be ligated. In another embodiment, the vector is a viral vector, wherein an additional DNA segment can be ligated into viral genome. The vectors disclosed herein are capable of self-replicating in a host cell into which they have been introduced (for example, a bacterial vector having a bacterial replication origin and a episomal mammalian vector) or can be integrated into the genome of a host cell upon introduction into host cell, thereby is replicated along with the host genome (e.g., a non-episomal mammalian vector).

The recombinant expression vector can be obtained by conventional methods in the art, that is, by connecting the nucleic acid molecule of the present disclosure to various expression vectors, thus being constructed. The expression vector is one of a variety of conventional vectors in the art, as long as it can carry the above-mentioned nucleic acid molecule. The vector preferably includes: various plasmids, cosmids, phage or virus vectors and the like.

The term “transfectoma”, as used herein, includes recombinant eukaryotic host cell expressing the antibody, such as CHO cells, NS/0 cells, HEK293 cells, plant cells, or fungi, including yeast cells.

The sequence of the DNA molecule for the antibody or a fragment thereof according to the present disclosure can be obtained by conventional techniques, for example, methods such as PCR amplification or genomic library screening. In addition, the sequences encoding light chain and heavy chain can be fused together, to form a single-chain antibody.

Once a relevant sequence is obtained, the relevant sequence can be obtained in bulk using a recombination method. This is usually carried out by cloning the sequence into a vector, transforming a cell with the vector, and then separating the relevant sequence from the proliferated host cell by conventional methods.

In addition, a relevant sequence can be synthesized artificially, especially when the fragment is short in length. Usually, several small fragments are synthesized first, and then are linked together to obtain a fragment with a long sequence.

At present, it is possible to obtain a DNA sequence encoding the antibody of the present disclosure (or fragments thereof, or derivatives thereof) completely by chemical synthesis. The DNA sequence can then be introduced into a variety of existing DNA molecules (or, for example, vectors) and cells known in the art. In addition, mutations can also be introduced into the protein sequences of the present disclosure by chemical synthesis.

In general, under conditions suitable for expression of the antibody according to the present disclosure, the host cell obtained is cultured. Then, the antibody of the present disclosure is purified by using conventional immunoglobulin purification steps, for example, the conventional separation and purification means well known to those skilled in the art, such as protein A-Sepharose, hydroxyapatite chromatography, gel electrophoresis, dialysis, ion exchange chromatography, hydrophobic chromatography, molecular sieve chromatography or affinity chromatography.

The monoclonal antibody obtained can be identified by conventional means. For example, the binding specificity of a monoclonal antibody can be determined by immunoprecipitation or an in vitro binding assay (such as radioimmunoassay (RIA) or enzyme-linked immunosorbent assay (ELISA)). The binding affinity of a monoclonal antibody can be determined by, for example, the Scatchard analysis (Munson et al., Anal. Biochem., 107: 220 (1980)).

The antibody according to the present disclosure can be expressed in a cell or on the cell membrane, or is secreted extracellularly. If necessary, the recombinant protein can be separated and purified by various separation methods according to its physical, chemical, and other properties.

These methods are well known to those skilled in the art. The examples of these methods comprise, but are not limited to, conventional renaturation treatment, treatment by protein precipitant (such as salt precipitation), centrifugation, cell lysis by osmosis, ultrasonic treatment, supercentrifugation, molecular sieve chromatography (gel chromatography), adsorption chromatography, ion exchange chromatography, high performance liquid chromatography (HPLC), and any other liquid chromatography, and the combination thereof.

The term “identity” of sequences refers to the relationship between the sequences of two or more polypeptide molecules or two or more nucleic acid molecules, as determined by aligning sequences. Generally, identity refers to the number or percentage of identical positions shared by two amino acid or nucleic acid sequences, taking into account the number of gaps, and the length of each gap, which need to be introduced for optimal alignment of the two sequences. Generally, before calculating the percentage of identity between two amino acid or nucleotide sequences, sequence alignment is performed, and gap (if any) is introduced. If the amino acid residues or bases in the two sequences are the same at a certain alignment position, the two sequences are considered to be identical or matched at that position. If the amino acid residues or bases in the two sequences are different, it is considered that they are inconsistent or mismatched at this position. In some algorithms, the number of matching positions is divided by the total number of positions in the alignment window to obtain sequence identity. In other algorithms, the number of notches and/or the length of notches are also taken into account. For the purpose of the present disclosure, the well-known alignment software BLAST (which can be found on the web page ncbi.nlm.nih.gov) can be used to obtain the best sequence alignment and calculate the sequence identity between the two amino acid or nucleotide sequences by using the default settings. When an amino acid sequence is described as being at least 85% or at least 90% or at least 95% identical to another amino acid sequence, the difference in the amino acid sequence may lie in conservative substitution (including all substitutions therein are conservative substitutions).

The term “variant” of a polypeptide such as an antigen-binding fragment, a protein or an antibody is a polypeptide in which one or more amino acid residues are inserted, deleted, added and/or substituted, as compared to another polypeptide sequence, and includes a fusion polypeptide. In addition, a protein variant includes the one modified by enzyme digestion, phosphorylation or other posttranslational modification, but maintaining biological activity of the antibody disclosed herein, for example, binding to CDH17 and specificity. The variant may be about 99%, 98%, 97%, 96%, 95%, 94%, 93%, 92%, 91%, 90%, 89%, 88%, 87%, 86%, 85%, 84%, 83%, 82%, 81%, or 80% identical to the sequence of the antibody or its antigen-binding fragment disclosed herein. The percent identity (%) or homology may be calculated with reference to the following description.

In the present disclosure, the antibody of the present disclosure also includes a conservative variant (conventional variants) thereof, which means that, compared to the amino acid sequence of the antibody of the present disclosure, there are up to 10, preferably up to 8 and more preferably up to 5, most preferably up to 3 amino acids are replaced by amino acids with similar or similar properties to form a polypeptide. These conservative variant polypeptides are preferably produced by conservative substitutions according to Table A.

TABLE A

| Original residue | Representative replacement | Preferred replacement |
|---|---|---|
| Ala | Val; Leu; Ile | Val |
| Arg | Lys; Gln; Asn | Lys |
| Asn | Gln; His; Lys; Arg | Gln |
| Asp | Glu | Glu |
| Cys | Ser | Ser |
| Gln | Asn | Asn |
| Glu | Asp | Asp |
| Gly | Pro; Ala | Ala |
| His | Asn; Gln; Lys; Arg | Arg |
| Ile | Leu; Val; Met; Ala; Phe | Leu |
| Leu | Ile; Val; Met; Ala; Phe | Ile |
| Lys | Arg; Gln; Asn | Arg |
| Met | Leu; Phe; Ile | Leu |
| Phe | Leu; Val; Ile; Ala; Tyr | Leu |
| Pro | Ala | Ala |
| Ser | Thr | Thr |
| Thr | Ser | Ser |
| Trp | Tyr; Phe | Tyr |
| Tyr | Trp; Phe; Thr; Ser | Phe |
| Val | Ile; Leu; Met; Phe; Ala | Leu |

The term “K_{D}” (M), as used herein, is intended to refer to the dissociation equilibrium constant of a particular antibody-antigen interaction. “K_{D}” refers to the dissociation constant, which is obtained from the ratio of K_{d} to Kₐ (i.e., K_{d}/Kₐ) and is expressed as a molar concentration (M). K_{D} values for antibodies can be determined using methods in the art in view of the present disclosure. For example, the K_{D} of an antibody can be determined by using surface plasmon resonance, such as by using a biosensor system, e.g., a system, or by using bio-layer interferometry technology, such as an Octet RED96 system.

The term “affinity” is the strength of interaction between an antibody or its antigen-binding fragment and an antigen, and it is determined by properties of the antigen such as size, structure and/or charge of antigen, and CDR sequences of the antibody or antigen-binding fragment. The methods for determining the affinity are known in the art, and the followings can be referred.

The antibody or its antigen-binding fragment is deemed to “specifically bind” to its target such as an antigen, when a dissociation constant (K_{D}) is <10⁻⁶ M. The antibody specifically binds to a target with “high affinity”, when K_{D} is <10⁻⁹ M.

The term “Pharmaceutical composition”, as used herein, is intended to refer to a mixture containing one or more of the antibody (or antigen-binding fragment thereof) or a physiological/pharmaceutically acceptable salt or prodrug thereof described herein with other chemical components, such as physiological/pharmaceutically acceptable carriers and excipients. The purpose of the pharmaceutical composition is to promote the administration to the organism, which is beneficial to the absorption of the active ingredient and exerts the biological activity.

“Administration” and “treatment,” when applied to an animal, human, experimental subject, cell, tissue, organ, or biological fluid, refer to contact with an exogenous pharmaceutical, therapeutic, diagnostic reagent, or composition with the animal, human, subject, cell, tissue, organ, or biological fluid. “Administration” and “treatment” can refer, e.g., to therapeutic, pharmacokinetic, diagnostic, research, and experimental methods. Treatment of a cell encompasses contacting the cell with a reagent, as well as contacting a fluid with a reagent, wherein the fluid is in contact with the cell. “Administration” and “treatment” also mean in vitro and ex vivo treatments, e.g., of a cell, by a reagent, diagnostic, binding composition or by another cell. “Treatment,” when applied to a human, veterinary, or research subject, refers to therapeutic treatment, prophylactic or preventative measures, research and diagnostic applications.

In addition, the present disclosure includes a medicament for treating a disease associated with CDH17, comprising an antibody or an antigen-binding fragment thereof of the present disclosure as an active ingredient.

There is no limitation on the diseases related to CDH17, as long as it is a disease associated with CDH17, for example, the therapeutic response induced by the molecules disclosed in the present disclosure can be reduced by binding human CDH17. Therefore, the molecules of the present disclosure are very useful for those who suffer a tumor, cancer or infectious disease when in preparations and formulations suitable for therapeutic applications.

In addition, the present disclosure relates to a method for immunologically detecting or measuring CDH17, a reagent for immunologically detecting or measuring CDH17, a method for immunologically detecting or measuring cells expressing CDH17, and a diagnostic reagent for diagnosis of disease related to CDH17 positive cells, comprising the antibody or antigen-binding fragment of the present disclosure that specifically recognizes human CDH17, as an active ingredient.

In the present disclosure, the method for detecting or determining the amount of CDH17 may be any known method. For example, it includes immunodetection or assay.

The immunodetection or assay is a method of detecting or determining the amount of antibody or antigen by using labeled antigen or antibody. Examples of immunodetection or assay include a radioactive substance labeled immunological antibody method (RIA), an enzyme immunoassay (EIA or ELISA), a fluorescent immunoassay (FIA), a luminescent immunoassay, a western blotting method, physicochemical methods, etc.

The above-mentioned diseases related to CDH17 positive cells can be diagnosed by detecting or measuring cells expressing CDH17 by using the antibodies or antibody fragments thereof or the diagnostic conjugate of the present disclosure.

In order to detect cells expressing the polypeptide, a known immunodetection can be used, and preferably immunoprecipitation, fluorescent cell staining or immunohistochemical staining etc. can be used. Furthermore, a fluorescent antibody staining method etc. using FMAT8100HTS system (Applied Biosystem) can be used.

### EXAMPLES

The invention is further illustrated by the following specific examples. It is to be understood that these examples are for illustrative purposes only and are not intended to limit the scope of the invention. The experimental methods without detailed conditions in the following examples are generally in accordance with the conditions described in the conventional conditions such as Sambrook. J et al. “Guide to Molecular Cloning Laboratory” (translated by Huang Peitang et al., Beijing: Science Press, 2002), or in accordance with the conditions recommended by the manufacturer (for example, product manuals). Percentages and parts are by weight unless otherwise stated. The experimental materials and reagents used in the following examples are commercially available unless otherwise specified.

The room temperature described in the examples is a conventional room temperature in the art, and is generally 10-30° C.

### Example 1 Mouse Immunization and Hybridoma Fusion

### Preparation of Antigens

A combination of recombinant protein antigen human CDH17 His Tag (Sino biological, catalog number 11360-H08H) was used to immunize the mice.

### Immunization Protocol:

Anti-CDH17 antibodies were obtained by immunizing genetically modified mouse encoding human immunoglobulin heavy and kappa light chain variable regions with recombinant protein antigen human CDH17 His Tag (Sino biological, catalog number 11360-H08H) and boosted with the same antigen. The antibody immune response was monitored by a CDH17 specific immunoassay. When a desired immune response was achieved, splenocytes were harvested from each mouse and fused with mouse myeloma cells to preserve their viability and form hybridoma cells and screened for CDH17 specificity.

### Spleen Cell Fusion

The spleen lymphocytes and myeloma cells Sp2/0 (ATCC® CRL-158) were fused to obtain hybridoma cells by electrofusion or PEG fusion. PEG fusion was performed using Clonacell™ HY technology (STEMCELL technologies), following manufacturer's instructions. The primary cell: mouse myeloma cell line ratio was 1:1 for electrofusion, and 10:1 for PEG fusion.

### Example 2 Hybridoma Screening

Screening of hybridoma clones specifically binding to CDH17 proteins by ELISA ELISA was performed using the DuoSet ELISA Ancillary Kit (R&D System, DY008). ELISA plates were coated with 1 μg/ml of human CDH17 His Tag (Sino biological, catalog number 11360-H08H), or BSA overnight. Excess unbound proteins were washed off by washing the plates three times with the wash buffer before blocking for 1 hour at room temperature. 100 μl CDH17 hybridoma supernatant was added in each well and incubated for 1 hour at room temperature. Excess unbound antibodies were washed off and 100 μl of 1:30000 diluted secondary antibody Goat anti-mouse IgG Fc-HRP (ab5870) was added to each well for another 1 hour. Plates were washed before the addition 50 μL of chemiluminescence agents (color A and color B) according to manufacturer's protocol. The reactions were terminated using 25 μL of stop solution. Optical density at 450 nm of samples was measured by a microplate reader (PerkinElmer). All tested clones bound selectively to human CDH17 but not BSA.

TABLE 1
Binding characterization of hybridoma clones
to CDH17 protein by ELISA assay (OD450)

| | Protein binding | Protein binding |
|---|---|---|
| Clone ID | (human CDH17) | (BSA) |
| 1H10 | 1.7895 | 0.0399 |
| 2F9 | 1.7237 | 0.0718 |
| 4F9 | 1.5202 | 0.051 |
| 9E2 | 1.4804 | 0.0429 |
| 10E11 | 1.4809 | 0.0475 |
| 13C7 | 1.6185 | 0.0432 |
| 14B12 | 1.8849 | 0.041 |
| 15A4 | 1.6026 | 0.0431 |
| 17F3 | 1.663 | 0.0425 |
| 19C7 | 1.8366 | 0.0468 |
| 20B4 | 1.8098 | 0.0406 |

### Screening of Hybridoma Clones Specifically Binding to CDH17 Expressing Cancer Cells by Flow Cytometry

Hybridoma supernatants was subjected to binding tests on CDH17 positive cell line AsPC1 (ATCC, CRL-1682), GP2d (Creative bioarray, CSC-J9456), and CDH17 negative cell line SW480 (ATCC, CCL-228) using flow cytometry analysis. Briefly, 50 μL of cells in cell staining buffer (2×10⁶ cells/mL) was mixed with 50 μL undiluted supernatants. The mixture was incubated on ice for 1 hour and then washed with ice cold staining buffer twice. The cells were subsequently stained with 50 μL of PE labelled secondary antibody (1:250 dilution, Biolegend, Cat #405307) for 20 min. After washing by staining buffer and fixing by 4% PFA, cells were analyzed by flow cytometry. A purified anti-human CDH17 antibody was used as positive control (Sino Biological, Cat #11360-MM02). A purified mouse IgG1 antibody was used as isotype control (Biolegend, Cat #400102).

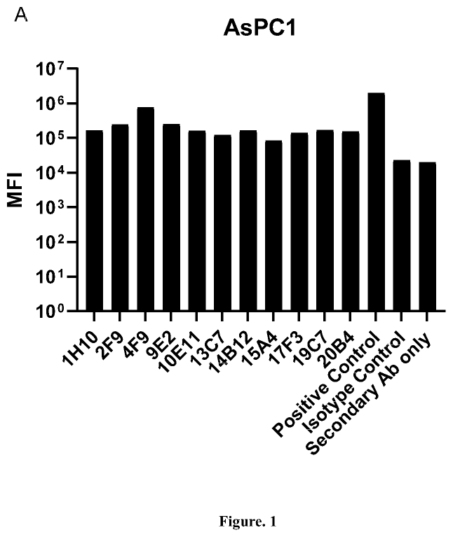

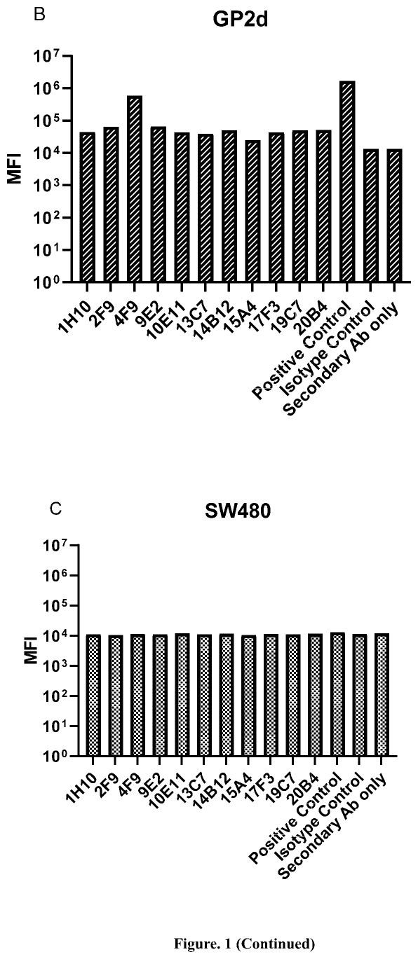

FIG. 1 shows examples of selected cell binding signals measured by flow cytometry. The hybridoma clones 1H10, 2F9, 4F9, 9E2, 10E11, 13C7, 14B12, 15A4, 17F3, 19C7, 20B4 were identified with enhanced binding profile to AsPC1 cells and GP2d cells compared to SW480 cells.

Screening of Hybridoma Clones with Cell Internalization Activity by Indirect Killing Assay

Cell internalization activity of hybridoma supernatants were measured using an indirect killing assay in AsPC1 cells. The AsPC1 cells were seeded on 96 well plate at 8,000 cells/well with propidium iodide (abeam, Cat #ab14083) at 500 ng/mL and SPY650-DNA (Cytoskeleton, Inc., Cat #CYSC501) at 1:2000 dilution. Cells were incubated overnight at 37° C. in 5% CO₂. The hybridoma supernatants from each hybridoma clone were diluted with hybridoma culture medium containing 500 ng/mL propidium iodide and 2000-fold diluted SPY650-DNA and mixed with Fab anti-mouse IgG Fc-MMAF conjugates with cleavable linker (Moradec, AM-202AF), then added to each well. The final concentrations of mouse IgG were roughly 10 nM, 3.33 nM, and 1.11 nM. The final concentration of the Fab anti-mouse IgG Fc-MMAF conjugates in each well was 20 nM. With the presence of secondary Fab-vc-MMAF, the internalized antibody/Fab-vc-MMAF conjugates complex will release the cytotoxic payload and kill the cells. Cytation5 (Agilent) was used to detect the viability of the cells in each well by imaging the plates every 8 h. A purified anti-human CDH17 antibody was used as positive control (Invitrogen, Cat #MA5-29135). A purified mouse IgG1 antibody was used as isotype control (Biolegend, Cat #400102).

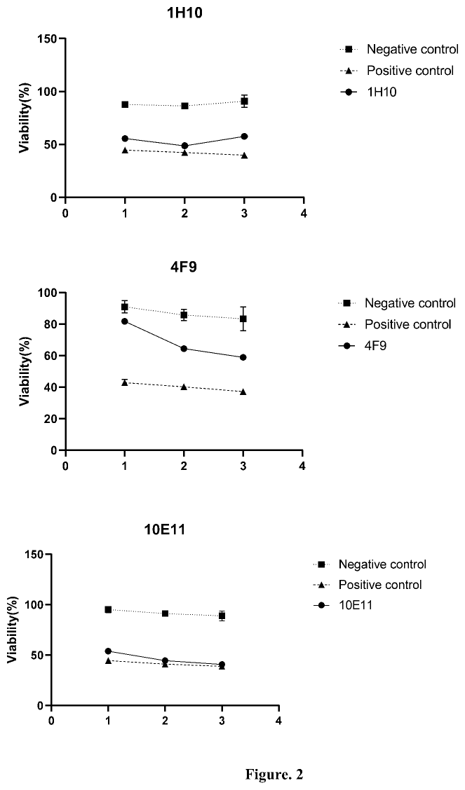

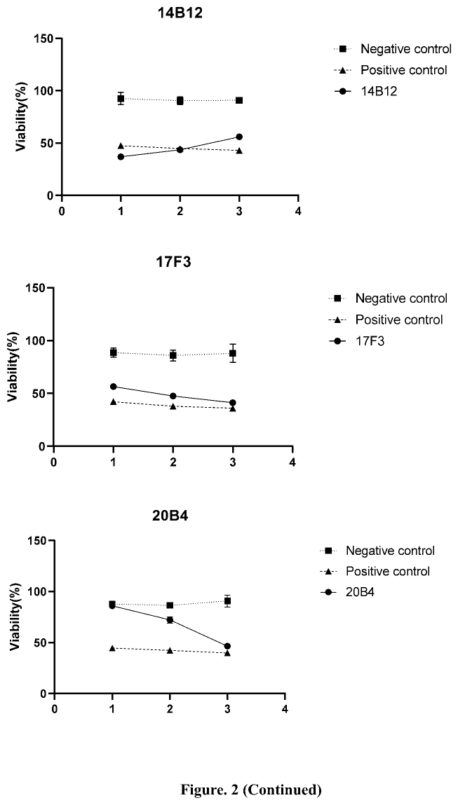

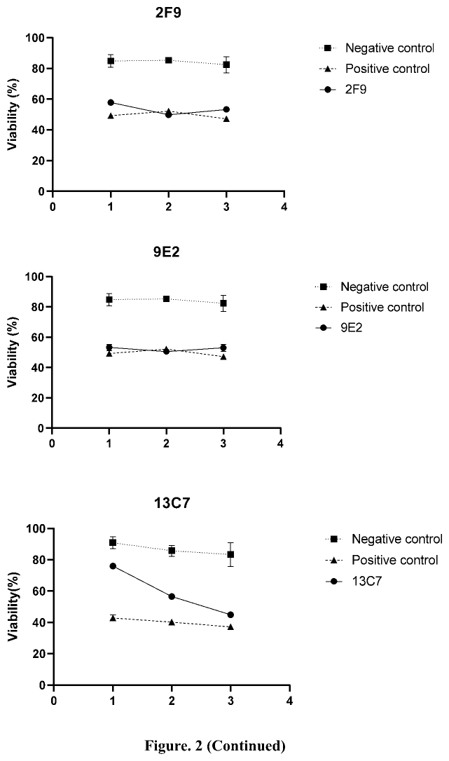

Cells treated with selected hybridoma supernatants showed reduced viability, as shown in FIG. 2, indicating the internalization of the antibodies. The clones 1H10, 2F9, 4F9, 9E2, 10E11, 13C7, 14B12, 15A4, 17F3, 19C7, 20B4 were identified to be capable of being internalized into CDH117 positive cell line using the indirect killing assay.

### Example 3 Sequencing of Positive Hybridoma Clones

The process of cloning sequences from positive hybridomas are as follows. The logarithmic growth phase hybridoma cells were collected, RNA was extracted, and reverse transcription was performed then followed by VDJ region amplification. Amplified cDNA library from each clone were subjected to next-generation sequencing. The amino acid sequences of the heavy and light chain variable region DNA sequences corresponding to the antibodies 1H10, 2F9, 4F9, 9F2, 10E11, 13C7, 14B12, 15A4, 17F3, 19C7, 20B4 were obtained. The amino acid sequence of heavy chain variable and light chain variable regions and CDR sequence of each antibody are as the following tables (Table 2 to Table 4). The amino acid residues of the CDRs in VH/VL are numbered and annotated according to the Kabat numbering system.

TABLE 2
CDR sequence of light chain variable domain for CDH17 hybridoma clones.

| | CDR Sequence of Heavy Chain Variable (VH) | | | | | |
|---|---|---|---|---|---|---|
| | CDR1 | | CDR2 | | CDR3 | |
| | | SEQ | | SEQ | | SEQ |
| | | ID | | ID | | ID |
| Clone | Sequence | NO | Sequence | NO | Sequence | NO |
| 1H10 | SYGMH | 1 | FIWYDGSNKNY | 11 | DRYSSGWDVF | 22 |
| | | | ADSVKG | | DY | |
| 2F9 | DYGMS | 2 | GLSWNGGNTY | 12 | GSRYNWNYD | 23 |
| | | | YADSVKG | | AFDI | |
| 4F9 | SSNWWS | 3 | EIYHSGSTSYNP | 13 | NDWGFPLDC | 24 |
| | | | SLKS | | | |
| 9E2 | NYGMS | 4 | GINWNGGSTYY | 14 | GSRYNWSYDA | 25 |
| | | | ADSVRG | | FDI | |
| 10E11 | SYWIG | 5 | IIYPGDSDTRYSP | 15 | RGWVNYFDY | 26 |
| | | | SFQG | | | |
| 13C7 | TSGVGVG | 6 | LIYWNDDKRYR | 16 | YGVTAPFYNW | 27 |
| | | | PSLKN | | FDP | |
| 14B12 | TYYRS | 7 | YIYYSGNTNYN | 17 | AQYGEEAFDI | 28 |
| | | | PSLKS | | | |
| 15A4 | SYGMH | 1 | FVWYDGSNQN | 18 | DRYSSGWDVF | 22 |
| | | | YADSVKG | | DY | |
| 17F3 | SYSLK | 8 | SISSSSGYIYYAD | 19 | AVSTIAARPGY | 29 |
| | | | SVKG | | YYYYMDV | |
| 19C7 | TGGVGVG | 9 | LIYWNDDKRYS | 20 | SGLAAPFYNW | 30 |
| | | | PSLKS | | FDP | |
| 20B4 | EVFMH | 10 | GFDPEDGETIYA | 21 | GWGSRYFDY | 31 |
| | | | QKFQG | | | |

TABLE 3
CDR sequence of light chain variable domain for CDH17 hybridoma clones.

| | CDR Sequence of Light Chain Variable (VL) | | | | | |
|---|---|---|---|---|---|---|
| | CDR1 | | CDR2 | | CDR3 | |
| | | SEQ ID | | SEQ ID | | SEQ ID |
| Clone | Sequence | NO | Sequence | NO | Sequence | NO |
| 1H10 | RASQDISSWLA | 32 | AASSLQS | 43 | QQAKSFPPT | 48 |
| 2F9 | RASQGIRNALG | 33 | AASNLQS | 44 | LQHNSYPFT | 49 |
| 4F9 | RASQSISSYLN | 34 | AASSLQS | 43 | QQSHNIPIT | 50 |
| 9E2 | RASQGVRNALG | 35 | AASSLQS | 43 | LQHNSYPFT | 49 |
| 10E11 | RASQGISNWLG | 36 | AASSLQS | 43 | QQANSFPLT | 51 |
| 13C7 | RASQSITSYLN | 37 | AASSLQS | 43 | QRSYSTLT | 52 |
| 14B12 | RASQSITTYLN | 38 | AASRLQT | 45 | QQSYNTPPT | 53 |
| 15A4 | RASQGISSWLA | 39 | AASSLQS | 43 | QQAKSFPPT | 48 |
| 17F3 | RASQDISTWFA | 40 | AASTLRS | 46 | QQANSFPLT | 51 |
| 19C7 | RASQSISNYLN | 41 | AASSLQS | 43 | QRSYSTLT | 52 |
| 20B4 | KSSQSVLYSSNN | 42 | WASTRES | 47 | HQYYSTPLT | 54 |
| | KNYLA | | | | | |

TABLE 4
Sequences of heavy chain and light chain variable domains for CDH17 hybridoma
clones.

| | Sequence of Heavy Chain Variable | | Sequence of Light Chain Variable | |
|---|---|---|---|---|
| | | SEQ ID | | SEQ ID |
| Clone | Sequence | NO | Sequence | NO |
| 1H10 | QVQLVESGGGVVQPGRS | 55 | DIQMTQSPSSVSASVGDRV | 66 |
| | LRLSCVASGFTFSSYGMH | | TITCRASQDISSWLAWYQQ | |
| | WVRQAPGKGLEWVTFI | | QPGKAPKLQIYAASSLQSG | |
| | WYDGSNKNYADSVKGR | | VPSRFSGSGSGTDFTLTISS | |
| | FTISRDNSKNTLNLQMNS | | LQPEDFATYYCQQAKSFPP | |
| | LRVEDTAVYYCARDRYSS | | TFGQGTKVEIK | |
| | GWDVFDYWGQGTLVTV | | | |
| | SS | | | |
| 2F9 | EVQLVESGGGVVRPGGS | 56 | DIQMTQSPSSLSASIGDRVT | 67 |
| | LRLSCAASGFTFDDYGM | | ITCRASQGIRNALGWYQQ | |
| | SWVRQAPGKGLEWVSG | | KPGKAPKRLIYAASNLQSG | |
| | LSWNGGNTYYADSVKGR | | VPSRFSGSGSGTEFTLTISSL | |
| | FTISRDNAKNSLYLQMNS | | QPEDFATYYCLQHNSYPFT | |
| | LRAEDTALYYCARGSRY | | FGPGTKVEIK | |
| | NWNYDAFDIWGQGTLVT | | | |
| | VSS | | | |
| 4F9 | QVQLQESGPGLVKPSGTL | 57 | DIQMTQSPSSLSASVGDRV | 68 |
| | SLTCAVSGGSISSSNWWS | | TITCRASQSISSYLNWYQQ | |
| | WVRQPPGKGLEWIGEIY | | KPGKAPKLLIYAASSLQSG | |
| | HSGSTSYNPSLKSRVTISV | | VPSRFSGSGSGTHFTLTISS | |
| | DKSKNQFSLKLSSVTAAD | | LQPEDFATYYCQQSHNIPIT | |
| | TAVYYCARNDWGFPLDC | | FGQGTRLEIK | |
| | WGQGTLVTVSS | | | |
| 9E2 | EVQLVESGGGVVRPGGS | 58 | DIQMTQSPSSLSASVGDRV | 69 |
| | LRLSCAASGFTFDNYGM | | TITCRASQGVRNALGWYQ | |
| | SWVRQAPGKGLEWVSGI | | QKPGKAPKRLIYAASSLQS | |
| | NWNGGSTYYADSVRGRF | | GVPSRFSGSGSGTEFTLTIS | |
| | TISRDNAKNSLYLQMNSL | | SLQPEDFATYYCLQHNSYP | |
| | RAEDTALYYCARGSRYN | | FTFGPGTKVEIK | |
| | WSYDAFDIWGQGTLVTV | | | |
| | SS | | | |
| 10E11 | EVQLVQSGTEVKKPGESL | 59 | DIQMTQSPSSVSASVGDRV | 70 |
| | KISCKDSGYRFSSYWIGW | | TITCRASQGISNWLGWYQ | |
| | VRQMPGKGLEWMGIIYP | | QKPGKAPKLLIYAASSLQS | |
| | GDSDTRYSPSFQGQVTIS | | GVPSRFSGSGSGTDFTLTIS | |
| | ADKSISTAYLQWRSLKAS | | SLQPEDFATYYCQQANSFP | |
| | DTAMYYCARRGWVNYF | | LTFGGGTKVEIK | |
| | DYWGQGTLVTVSS | | | |
| 13C7 | QITLKESGPTLVKPTQTLT | 60 | DIQMTQSPSSLSASVGDRV | 71 |
| | LTCTFSGFSLSTSGVGVG | | TITCRASQSITSYLNWYQQ | |
| | WIRQPPGKALEWLALIY | | KPGKAPKLLIYAASSLQSG | |
| | WNDDKRYRPSLKNRLTIT | | VPSRFSGSGSGTDFTLTISS | |
| | KDTSKNQVVLTMTNMDP | | LQPEDFATYYCQRSYSTLT | |
| | VDTATYYCTHYGVTAPF | | FGGGTKVEIK | |
| | YNWFDPWGQGTLVTVSS | | | |
| 14B12 | QVQLQESGPGLVKPSETL | 61 | DIQMTQSPSSLSASVGDRV | 72 |
| | SLTCTVSGGSINTYYRSW | | TITCRASQSITTYLNWYQQ | |
| | IRQPPGKGLEWIGYIYYS | | KPGKAPQLLIFAASRLQTG | |
| | GNTNYNPSLKSRVTISAD | | VPSRFSGSGSGTDFTLTISS | |
| | TSKNQFSLNLISVTAADT | | LLPGDFATYYCQQSYNTPP | |
| | AMYYCARAQYGEEAFDI | | TFGGGTKVEIK | |
| | WGQGTLVTVSS | | | |
| 15A4 | QVQLVESGGGVVQPGRS | 62 | DIQMTQSPSSVSASVGDRV | 73 |
| | LRLSCAASGFNFSSYGM | | TITCRASQGISSWLAWYQQ | |
| | HWVRQAPGKGLEWVAF | | KPGKAPKLLIFAASSLQSG | |
| | VWYDGSNQNYADSVKG | | VPSRFSGSGSGTGFTLTISS | |
| | RFTISRDNSNNTLYLQMN | | LQPEDFATYYCQQAKSFPP | |
| | SLRAEDTAVYYCARDRY | | TFGQGTKVEIK | |
| | SSGWDVFDYWGQGTLV | | | |
| | TVSS | | | |
| 17F3 | EVQLVESGGGLVKSGGSL | 63 | DIQMTQSPSSVSASIGDRV | 74 |
| | RLSCAASGFTFSSYSLKW | | TITCRASQDISTWFAWYQQ | |
| | VRQAPGKGLEWVSSISSS | | KPGKAPKLLIYAASTLRSG | |
| | SGYIYYADSVKGRFTISR | | VPSRFSGSGSGTDFTLTINS | |
| | DNAKNSLYLQMNSLRAE | | LQPEDFATYYCQQANSFPL | |
| | DTAMYYCARAVSTIAAR | | TFGGGTKVEIK | |
| | PGYYYYYMDVWGKGTL | | | |
| | VTVSS | | | |
| 19C7 | QITLKESGPTLVKPTQTLT | 64 | DIQMTQSPSSLSASVGDRV | 75 |
| | LTCTFSGISLSTGGVGVG | | TITCRASQSISNYLNWYQL | |
| | WIRQPPGKALEWLSLIY | | KPGNAPKLLIFAASSLQSG | |
| | WNDDKRYSPSLKSRLTIT | | VPSRFSGSGSGTDFTLTISS | |
| | KDSSKNQVVLTMTNMDP | | LQPEDFATYFCQRSYSTLTF | |
| | VDTATYYCAHSGLAAPF | | GGGTKVEIK | |
| | YNWFDPWGQGTLVTVSS | | | |
| 20B4 | QVQLVQSGAEVKKPGAS | 65 | DIVMTQSPDSLAVSLGERA | 76 |
| | VKVSCKVSGYTLTEVFM | | TINCKSSQSVLYSSNNKNY | |
| | HWVRQAPGKGLEWMGG | | LAWYQQKPGQPPKLLIYW | |
| | FDPEDGETIYAQKFQGRV | | ASTRESGVPDRFSGSGSGT | |
| | TMTEDTFIDTAYMDLSSL | | DFTLTISSLQAEDVAVYYC | |
| | RSEDTAVYYCAIGWGSR | | HQYYSTPLTFGGGTKVEIK | |
| | YFDYWGQGTLVTVSS | | | |

### Example 4 Expression, Purification and Binding Characterization of Recombinant Antibody

### Molecular Cloning of Recombinant Antibodies

The cDNA sequences that encode VH and VL regions of selected clones were directly synthesized as DNA fragments with 5′-end in-frame leader sequence (MGWSCIILFLVATATGVHS). These DNA fragments were cloned into selected vectors using NEBuilder DNA Assembly Cloning Kit (New England Biolabs). VH region was cloned into pFUSE-CHIg_hG1 vector (InvivoGen #pfuse-hchg1), which in-frame with constant region of hIgG1 heavy chain in the vector. VL region was cloned into pFUSE2-CLIg_hk vector (InvivoGen, #pfuse2-hclk), which in-frame with constant region of hIg kappa light chain in the vector. The amino acid sequences of the constant region of hIgG1 heavy chain and the constant region of hIg kappa light chain are as follow:

>Heavy chain constant region (SEQ ID NO: 99):
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQ
QGNVFSCSVMHEALHNHYTQKSLSLSPGK;
>Light chain constant region (SEQ ID NO: 100):
RTVAAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC.

The IgG form of antibodies were disclosed as the following heavy chain and light chain full-lengths in Table 5. 1H10: SEQ ID NO: 77 (heavy chain) and 78 (light chain); 2F9: SEQ ID NO 79 (heavy chain) and 80 (light chain); 4F9: SEQ ID NO 81 (heavy chain) and 82 (light chain); 9E2: SEQ ID NO 83 (heavy chain) and 84 (light chain); 10E11: SEQ ID NO: 85 (heavy chain) and 86 (light chain); 13C7: SEQ ID NO 87 (heavy chain) and 88 (light chain); 14B12: SEQ ID NO 89 (heavy chain) and 90 (light chain); 15A4: SEQ ID NO 91 (heavy chain) and 92 (light chain); 17F3: SEQ ID NO 93 (heavy chain) and 94 (light chain); 19CT: SEQ ID NO: 95 (heavy chain) and 96 (light chain); 20B4: SEQ ID NO 97 (heavy chain) and 98 (light chain).

TABLE 5
Sequences of heavy chain and light chain full lengths for anti-CDH17 recombinant
antibodies

| | Sequence of Heavy Chain | | Sequence of Light Chain | |
|---|---|---|---|---|
| | | SEQ | | SEQ |
| Clone | Sequence | ID NO | Sequence | ID NO |
| 1H10 | QVQLVESGGGVVQPGRSLRLSCVASGFTFSS | 77 | DIQMTQSPSSVSASVGDR | 78 |
| | YGMHWVRQAPGKGLEWVTFIWYDGSNKN | | VTITCRASQDISSWLAWY | |
| | YADSVKGRFTISRDNSKNTLNLQMNSLRVE | | QQQPGKAPKLQIYAASSL | |
| | DTAVYYCARDRYSSGWDVFDYWGQGTLVT | | QSGVPSRFSGSGSGTDFTL | |
| | VSSASTKGPSVFPLAPSSKSTSGGTAALGCL | | TISSLQPEDFATYYCQQAK | |
| | VKDYFPEPVTVSWNSGALTSGVHTFPAVLQ | | SFPPTFGQGTKVEIKRTVA | |
| | SSGLYSLSSVVTVPSSSLGTQTYICNVNHKP | | APSVFIFPPSDEQLKSGTAS | |
| | SNTKVDKKVEPKSCDKTHTCPPCPAPELLG | | VVCLLNNFYPREAKVQW | |
| | GPSVFLFPPKPKDTLMISRTPEVTCVVVDVS | | KVDNALQSGNSQESVTEQ | |
| | HEDPEVKFNWYVDGVEVHNAKTKPREEQY | | DSKDSTYSLSSTLTLSKAD | |
| | NSTYRVVSVLTVLHQDWLNGKEYKCKVSN | | YEKHKVYACEVTHQGLSS | |
| | KALPAPIEKTISKAKGQPREPQVYTLPPSREE | | PVTKSFNRGEC | |
| | MTKNQVSLTCLVKGFYPSDIAVEWESNGQP | | | |
| | ENNYKTTPPVLDSDGSFFLYSKLTVDKSRW | | | |
| | QQGNVFSCSVMHEALHNHYTQKSLSLSPGK | | | |
| 2F9 | EVQLVESGGGVVRPGGSLRLSCAASGFTFD | 79 | DIQMTQSPSSLSASIGDRV | 80 |
| | DYGMSWVRQAPGKGLEWVSGLSWNGGNT | | TITCRASQGIRNALGWYQ | |
| | YYADSVKGRFTISRDNAKNSLYLQMNSLRA | | QKPGKAPKRLIYAASNLQ | |
| | EDTALYYCARGSRYNWNYDAFDIWGQGTL | | SGVPSRFSGSGSGTEFTLTI | |
| | VTVSSASTKGPSVFPLAPSSKSTSGGTAALG | | SSLQPEDFATYYCLQHNS | |
| | CLVKDYFPEPVTVSWNSGALTSGVHTFPAVL | | YPFTFGPGTKVEIKRTVAA | |
| | QSSGLYSLSSVVTVPSSSLGTQTYICNVNHK | | PSVFIFPPSDEQLKSGTASV | |
| | PSNTKVDKKVEPKSCDKTHTCPPCPAPELLG | | VCLLNNFYPREAKVQWK | |
| | GPSVFLFPPKPKDTLMISRTPEVTCVVVDVS | | VDNALQSGNSQESVTEQD | |
| | HEDPEVKFNWYVDGVEVHNAKTKPREEQY | | SKDSTYSLSSTLTLSKADY | |
| | NSTYRVVSVLTVLHQDWLNGKEYKCKVSN | | EKHKVYACEVTHQGLSSP | |
| | KALPAPIEKTISKAKGQPREPQVYTLPPSREE | | VTKSFNRGEC | |
| | MTKNQVSLTCLVKGFYPSDIAVEWESNGQP | | | |
| | ENNYKTTPPVLDSDGSFFLYSKLTVDKSRW | | | |
| | QQGNVFSCSVMHEALHNHYTQKSLSLSPGK | | | |
| 4F9 | QVQLQESGPGLVKPSGTLSLTCAVSGGSISSS | 81 | DIQMTQSPSSLSASVGDRV | 82 |
| | NWWSWVRQPPGKGLEWIGEIYHSGSTSYN | | TITCRASQSISSYLNWYQQ | |
| | PSLKSRVTISVDKSKNQFSLKLSSVTAADTA | | KPGKAPKLLIYAASSLQSG | |
| | VYYCARNDWGFPLDCWGQGTLVTVSSAST | | VPSRFSGSGSGTHFTLTISS | |
| | KGPSVFPLAPSSKSTSGGTAALGCLVKDYFP | | LQPEDFATYYCQQSHNIPI | |
| | EPVTVSWNSGALTSGVHTFPAVLQSSGLYSL | | TFGQGTRLEIKRTVAAPSV | |
| | SSVVTVPSSSLGTQTYICNVNHKPSNTKVDK | | FIFPPSDEQLKSGTASVVC | |
| | KVEPKSCDKTHTCPPCPAPELLGGPSVFLFP | | LLNNFYPREAKVQWKVD | |
| | PKPKDTLMISRTPEVTCVVVDVSHEDPEVKF | | NALQSGNSQESVTEQDSK | |
| | NWYVDGVEVHNAKTKPREEQYNSTYRVVS | | DSTYSLSSTLTLSKADYEK | |
| | VLTVLHQDWLNGKEYKCKVSNKALPAPIEK | | HKVYACEVTHQGLSSPVT | |
| | TISKAKGQPREPQVYTLPPSREEMTKNQVSL | | KSFNRGEC | |
| | TCLVKGFYPSDIAVEWESNGQPENNYKTTPP | | | |
| | VLDSDGSFFLYSKLTVDKSRWQQGNVFSCS | | | |
| | VMHEALHNHYTQKSLSLSPGK | | | |
| 9E2 | EVQLVESGGGVVRPGGSLRLSCAASGFTFD | 83 | DIQMTQSPSSLSASVGDRV | 84 |
| | NYGMSWVRQAPGKGLEWVSGINWNGGST | | TITCRASQGVRNALGWYQ | |
| | YYADSVRGRFTISRDNAKNSLYLQMNSLRA | | QKPGKAPKRLIYAASSLQS | |
| | EDTALYYCARGSRYNWSYDAFDIWGQGTL | | GVPSRFSGSGSGTEFTLTIS | |
| | VTVSSASTKGPSVFPLAPSSKSTSGGTAALG | | SLQPEDFATYYCLQHNSY | |
| | CLVKDYFPEPVTVSWNSGALTSGVHTFPAVL | | PFTFGPGTKVEIKRTVAAP | |
| | QSSGLYSLSSVVTVPSSSLGTQTYICNVNHK | | SVFIFPPSDEQLKSGTASV | |
| | PSNTKVDKKVEPKSCDKTHTCPPCPAPELLG | | VCLLNNFYPREAKVQWK | |
| | GPSVFLFPPKPKDTLMISRTPEVTCVVVDVS | | VDNALQSGNSQESVTEQD | |
| | HEDPEVKFNWYVDGVEVHNAKTKPREEQY | | SKDSTYSLSSTLTLSKADY | |
| | NSTYRVVSVLTVLHQDWLNGKEYKCKVSN | | EKHKVYACEVTHQGLSSP | |
| | KALPAPIEKTISKAKGQPREPQVYTLPPSREE | | VTKSFNRGEC | |
| | MTKNQVSLTCLVKGFYPSDIAVEWESNGQP | | | |
| | ENNYKTTPPVLDSDGSFFLYSKLTVDKSRW | | | |
| | QQGNVFSCSVMHEALHNHYTQKSLSLSPGK | | | |
| 10E11 | EVQLVQSGTEVKKPGESLKISCKDSGYRFSS | 85 | DIQMTQSPSSVSASVGDR | 86 |
| | YWIGWVRQMPGKGLEWMGIIYPGDSDTRY | | VTITCRASQGISNWLGWY | |
| | SPSFQGQVTISADKSISTAYLQWRSLKASDT | | QQKPGKAPKLLIYAASSL | |
| | AMYYCARRGWVNYFDYWGQGTLVTVSSA | | QSGVPSRFSGSGSGTDFTL | |
| | STKGPSVFPLAPSSKSTSGGTAALGCLVKDY | | TISSLQPEDFATYYCQQAN | |
| | FPEPVTVSWNSGALTSGVHTFPAVLQSSGLY | | SFPLTFGGGTKVEIKRTVA | |
| | SLSSVVTVPSSSLGTQTYICNVNHKPSNTKV | | APSVFIFPPSDEQLKSGTAS | |
| | DKKVEPKSCDKTHTCPPCPAPELLGGPSVFL | | VVCLLNNFYPREAKVQW | |
| | FPPKPKDTLMISRTPEVTCVVVDVSHEDPEV | | KVDNALQSGNSQESVTEQ | |
| | KFNWYVDGVEVHNAKTKPREEQYNSTYRV | | DSKDSTYSLSSTLTLSKAD | |
| | VSVLTVLHQDWLNGKEYKCKVSNKALPAPI | | YEKHKVYACEVTHQGLSS | |
| | EKTISKAKGQPREPQVYTLPPSREEMTKNQ | | PVTKSFNRGEC | |
| | VSLTCLVKGFYPSDIAVEWESNGQPENNYK | | | |
| | TTPPVLDSDGSFFLYSKLTVDKSRWQQGNV | | | |
| | FSCSVMHEALHNHYTQKSLSLSPGK | | | |
| 13C7 | QITLKESGPTLVKPTQTLTLTCTFSGFSLSTSG | 87 | DIQMTQSPSSLSASVGDRV | 88 |
| | VGVGWIRQPPGKALEWLALIYWNDDKRYR | | TITCRASQSITSYLNWYQQ | |
| | PSLKNRLTITKDTSKNQVVLTMTNMDPVDT | | KPGKAPKLLIYAASSLQSG | |
| | ATYYCTHYGVTAPFYNWFDPWGQGTLVTV | | VPSRFSGSGSGTDFTLTISS | |
| | SSASTKGPSVFPLAPSSKSTSGGTAALGCLV | | LQPEDFATYYCQRSYSTLT | |
| | KDYFPEPVTVSWNSGALTSGVHTFPAVLQSS | | FGGGTKVEIKRTVAAPSVF | |
| | GLYSLSSVVTVPSSSLGTQTYICNVNHKPSN | | IFPPSDEQLKSGTASVVCL | |
| | TKVDKKVEPKSCDKTHTCPPCPAPELLGGPS | | LNNFYPREAKVQWKVDN | |
| | VFLFPPKPKDTLMISRTPEVTCVVVDVSHED | | ALQSGNSQESVTEQDSKD | |
| | PEVKFNWYVDGVEVHNAKTKPREEQYNST | | STYSLSSTLTLSKADYEKH | |
| | YRVVSVLTVLHQDWLNGKEYKCKVSNKAL | | KVYACEVTHQGLSSPVTK | |
| | PAPIEKTISKAKGQPREPQVYTLPPSREEMT | | SFNRGEC | |
| | KNQVSLTCLVKGFYPSDIAVEWESNGQPEN | | | |
| | NYKTTPPVLDSDGSFFLYSKLTVDKSRWQQ | | | |
| | GNVFSCSVMHEALHNHYTQKSLSLSPGK | | | |
| 14B12 | QVQLQESGPGLVKPSETLSLTCTVSGGSINT | 89 | DIQMTQSPSSLSASVGDRV | 90 |
| | YYRSWIRQPPGKGLEWIGYIYYSGNTNYNP | | TITCRASQSITTYLNWYQQ | |
| | SLKSRVTISADTSKNQFSLNLISVTAADTAM | | KPGKAPQLLIFAASRLQTG | |
| | YYCARAQYGEEAFDIWGQGTLVTVSSASTK | | VPSRFSGSGSGTDFTLTISS | |
| | GPSVFPLAPSSKSTSGGTAALGCLVKDYFPE | | LLPGDFATYYCQQSYNTP | |
| | PVTVSWNSGALTSGVHTFPAVLQSSGLYSLS | | PTFGGGTKVEIKRTVAAPS | |
| | SVVTVPSSSLGTQTYICNVNHKPSNTKVDK | | VFIFPPSDEQLKSGTASVV | |
| | KVEPKSCDKTHTCPPCPAPELLGGPSVFLFP | | CLLNNFYPREAKVQWKV | |
| | PKPKDTLMISRTPEVTCVVVDVSHEDPEVKF | | DNALQSGNSQESVTEQDS | |
| | NWYVDGVEVHNAKTKPREEQYNSTYRVVS | | KDSTYSLSSTLTLSKADYE | |
| | VLTVLHQDWLNGKEYKCKVSNKALPAPIEK | | KHKVYACEVTHQGLSSPV | |
| | TISKAKGQPREPQVYTLPPSREEMTKNQVSL | | TKSFNRGEC | |
| | TCLVKGFYPSDIAVEWESNGQPENNYKTTPP | | | |
| | VLDSDGSFFLYSKLTVDKSRWQQGNVFSCS | | | |
| | VMHEALHNHYTQKSLSLSPGK | | | |
| 15A4 | QVQLVESGGGVVQPGRSLRLSCAASGFNFS | 91 | DIQMTQSPSSVSASVGDR | 92 |
| | SYGMHWVRQAPGKGLEWVAFVWYDGSNQ | | VTITCRASQGISSWLAWY | |
| | NYADSVKGRFTISRDNSNNTLYLQMNSLRA | | QQKPGKAPKLLIFAASSLQ | |
| | EDTAVYYCARDRYSSGWDVFDYWGQGTLV | | SGVPSRFSGSGSGTGFTLTI | |
| | TVSSASTKGPSVFPLAPSSKSTSGGTAALGC | | SSLQPEDFATYYCQQAKSF | |
| | LVKDYFPEPVTVSWNSGALTSGVHTFPAVL | | PPTFGQGTKVEIKRTVAAP | |
| | QSSGLYSLSSVVTVPSSSLGTQTYICNVNHK | | SVFIFPPSDEQLKSGTASV | |
| | PSNTKVDKKVEPKSCDKTHTCPPCPAPELLG | | VCLLNNFYPREAKVQWK | |
| | GPSVFLFPPKPKDTLMISRTPEVTCVVVDVS | | VDNALQSGNSQESVTEQD | |
| | HEDPEVKFNWYVDGVEVHNAKTKPREEQY | | SKDSTYSLSSTLTLSKADY | |
| | NSTYRVVSVLTVLHQDWLNGKEYKCKVSN | | EKHKVYACEVTHQGLSSP | |
| | KALPAPIEKTISKAKGQPREPQVYTLPPSREE | | VTKSFNRGEC | |
| | MTKNQVSLTCLVKGFYPSDIAVEWESNGQP | | | |
| | ENNYKTTPPVLDSDGSFFLYSKLTVDKSRW | | | |
| | QQGNVFSCSVMHEALHNHYTQKSLSLSPGK | | | |
| 17F3 | EVQLVESGGGLVKSGGSLRLSCAASGFTFSS | 93 | DIQMTQSPSSVSASIGDRV | 94 |
| | YSLKWVRQAPGKGLEWVSSISSSSGYIYYA | | TITCRASQDISTWFAWYQ | |
| | DSVKGRFTISRDNAKNSLYLQMNSLRAEDT | | QKPGKAPKLLIYAASTLRS | |
| | AMYYCARAVSTIAARPGYYYYYMDVWGK | | GVPSRFSGSGSGTDFTLTI | |
| | GTLVTVSSASTKGPSVFPLAPSSKSTSGGTA | | NSLQPEDFATYYCQQANS | |
| | ALGCLVKDYFPEPVTVSWNSGALTSGVHTF | | FPLTFGGGTKVEIKRTVAA | |
| | PAVLQSSGLYSLSSVVTVPSSSLGTQTYICNV | | PSVFIFPPSDEQLKSGTASV | |
| | NHKPSNTKVDKKVEPKSCDKTHTCPPCPAP | | VCLLNNFYPREAKVQWK | |
| | ELLGGPSVFLFPPKPKDTLMISRTPEVTCVV | | VDNALQSGNSQESVTEQD | |
| | VDVSHEDPEVKFNWYVDGVEVHNAKTKPR | | SKDSTYSLSSTLTLSKADY | |
| | EEQYNSTYRVVSVLTVLHQDWLNGKEYKC | | EKHKVYACEVTHQGLSSP | |
| | KVSNKALPAPIEKTISKAKGQPREPQVYTLP | | VTKSFNRGEC | |
| | PSREEMTKNQVSLTCLVKGFYPSDIAVEWES | | | |
| | NGQPENNYKTTPPVLDSDGSFFLYSKLTVD | | | |
| | KSRWQQGNVFSCSVMHEALHNHYTQKSLS | | | |
| | LSPGK | | | |
| 19C7 | QITLKESGPTLVKPTQTLTLTCTFSGISLSTGG | 95 | DIQMTQSPSSLSASVGDRV | 96 |
| | VGVGWIRQPPGKALEWLSLIYWNDDKRYS | | TITCRASQSISNYLNWYQL | |
| | PSLKSRLTITKDSSKNQVVLTMTNMDPVDT | | KPGNAPKLLIFAASSLQSG | |
| | ATYYCAHSGLAAPFYNWFDPWGQGTLVTV | | VPSRFSGSGSGTDFTLTISS | |
| | SSASTKGPSVFPLAPSSKSTSGGTAALGCLV | | LQPEDFATYFCQRSYSTLT | |
| | KDYFPEPVTVSWNSGALTSGVHTFPAVLQSS | | FGGGTKVEIKRTVAAPSVF | |
| | GLYSLSSVVTVPSSSLGTQTYICNVNHKPSN | | IFPPSDEQLKSGTASVVCL | |
| | TKVDKKVEPKSCDKTHTCPPCPAPELLGGPS | | LNNFYPREAKVQWKVDN | |
| | VFLFPPKPKDTLMISRTPEVTCVVVDVSHED | | ALQSGNSQESVTEQDSKD | |
| | PEVKFNWYVDGVEVHNAKTKPREEQYNST | | STYSLSSTLTLSKADYEKH | |
| | YRVVSVLTVLHQDWLNGKEYKCKVSNKAL | | KVYACEVTHQGLSSPVTK | |
| | PAPIEKTISKAKGQPREPQVYTLPPSREEMT | | SFNRGEC | |
| | KNQVSLTCLVKGFYPSDIAVEWESNGQPEN | | | |
| | NYKTTPPVLDSDGSFFLYSKLTVDKSRWQQ | | | |
| | GNVFSCSVMHEALHNHYTQKSLSLSPGK | | | |
| 20B4 | QVQLVQSGAEVKKPGASVKVSCKVSGYTLT | 97 | DIVMTQSPDSLAVSLGER | 98 |
| | EVFMHWVRQAPGKGLEWMGGFDPEDGETI | | ATINCKSSQSVLYSSNNKN | |
| | YAQKFQGRVTMTEDTFIDTAYMDLSSLRSE | | YLAWYQQKPGQPPKLLIY | |
| | DTAVYYCAIGWGSRYFDYWGQGTLVTVSS | | WASTRESGVPDRFSGSGS | |
| | ASTKGPSVFPLAPSSKSTSGGTAALGCLVKD | | GTDFTLTISSLQAEDVAVY | |
| | YFPEPVTVSWNSGALTSGVHTFPAVLQSSGL | | YCHQYYSTPLTFGGGTKV | |
| | YSLSSVVTVPSSSLGTQTYICNVNHKPSNTK | | EIKRTVAAPSVFIFPPSDEQ | |
| | VDKKVEPKSCDKTHTCPPCPAPELLGGPSVF | | LKSGTASVVCLLNNFYPR | |
| | LFPPKPKDTLMISRTPEVTCVVVDVSHEDPE | | EAKVQWKVDNALQSGNS | |
| | VKFNWYVDGVEVHNAKTKPREEQYNSTYR | | QESVTEQDSKDSTYSLSST | |
| | VVSVLTVLHQDWLNGKEYKCKVSNKALPA | | LTLSKADYEKHKVYACEV | |
| | PIEKTISKAKGQPREPQVYTLPPSREEMTKN | | THQGLSSPVTKSFNRGEC | |
| | QVSLTCLVKGFYPSDIAVEWESNGQPENNY | | | |
| | KTTPPVLDSDGSFFLYSKLTVDKSRWQQGN | | | |
| | VFSCSVMHEALHNHYTQKSLSLSPGK | | | |

### Expression and Purification of Recombinant Antibodies

The heavy chain expression plasmid and light chain plasmids were co-transfected into CHO cells (ATCC, Cat #CCL-61) using ExpiFectamine 293 Transfection Kit (ThermoFisher, A14524), or into ExpiCHO-S cells (ThermoFisher #A29127) using ExpiFectamine CHO Transfection Kit (ThermoFisher, A29129). Based on the manufacturer's instructions, plasmid DNA concentration reached 1.0 μg per ml of suspended cells, with LC:HC vector ratio 1:1. The transfected cells were cultured 5 to 7 days on an orbital shaker at 37 C, 8% CO₂. Conditioned medium was collected and antibodies were purified using HiTrap MabSelect SuRe column (Cytiva, #17549112) on AKTAPure 25 machine (Cytiva). Eluted antibodies were neutralized with Tris Buffer (pH 9.0) and subjected to PBS buffer exchange. Product concentration was measured by UV absorption, and quality was determined by SDS-PAGE and HPLC.

### Binding Characterization of Anti-CDH17 Recombinant Antibody to CDH17 Positive and CDH17 Negative Cell Lines by Flow Cytometry

Binding of the recombinant antibody (human IgG1) to the cell surface CDH17 was determined by FACS analysis using cancer cell lines including CDH17 positive cancer cell AsPC1 cells and CDH17 negative cancer cell SW480 cells.

AsPC1 cells were maintained in RPMI-1640 medium supplemented with 10% FBS and 1% penicillin and streptomycin. SW480 cells were maintained in DMEM medium supplemented with 10% FBS and 1% penicillin and streptomycin. Cells were cultured at 37° C. with 5% CO₂ in humidified atmosphere.

To determine the binding of recombinant antibody to cell surface CDH17 receptors, cells were first harvested and resuspended in cell staining buffer (BioLegend, Cat #420201) at 1.3-1.5×10⁶ cells/mL. Then, the cells were treated with human Fc receptor blocking reagent (BioLegend, Cat #422302) on the ice for 10 min. The resulting cell suspension was aliquoted into 50 μL aliquots. 25 μL of recombinant antibody at various concentrations were mixed with the cell aliquots, the final concentration of recombinant antibody in the mixture ranged from 1.1 pM to 200 nM. The cells were incubated on the ice for 1 hour and then washed with cell staining buffer twice. 50 μL of secondary antibody (PE conjugated goat anti-human Fc, eBioscience™, 1:250 dilution) was added to each sample to resuspend the cells. The cells were incubated on the ice for another 20 min. Cells were subsequently washed twice with cell staining buffer and resuspended in 4% PFA to fix the cells. The samples were analyzed using iQue3 to measure the median fluorescence intensity using corresponding channels.

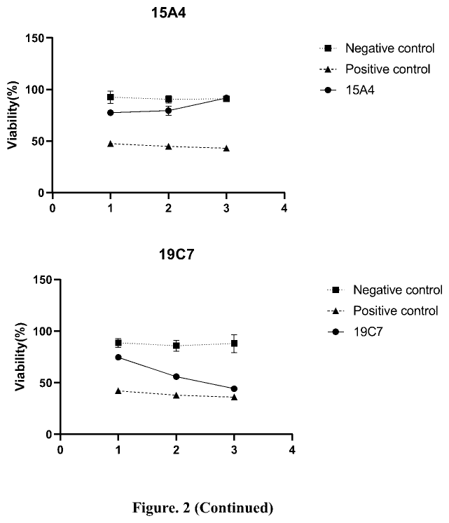

The result was disclosed in FIG. 3 and Table 6. HBMAB81 is a positive control monoclonal antibody disclosed in patent application WO2018115231A2. B12 is an in-house isotype control antibody disclosed in patent US005652138A. As a result, it was confirmed that the anti-CDH17 antibodies of the present disclosure specifically bind to the human CDH17 as originally expressed in cells in a concentration-dependent pattern, while HBMAB81 non-specifically binds to CDH17-negative cell line SW480.

TABLE 6
K_{D} values of anti-CDH17 recombinant antibodies
binding to CDH17 positive AsPC1 cells

| Antibody | K_{D} (nM) |
|---|---|
| 1H10 | 0.31 |
| 2F9 | 0.17 |
| 4F9 | 1.69 |
| 9E2 | 0.31 |
| 10E11 | 0.10 |
| 13C7 | 0.28 |
| 14B12 | 0.07 |
| 15A4 | 2.32 |
| 17F3 | 0.74 |
| 19C7 | 0.13 |
| 20B4 | 0.10 |

### Example 5 Characterization of Anti-CDH17 Recombinant Antibody Cell Internalization in CDH17 Expression Cells by Indirect Killing Assay

To evaluate the endocytosis of the antibodies induced by anti-CDH17 antibodies binding, an indirect killing assay was used to characterize the antibody internalization. Fab anti-human IgG Fe-MMAF conjugates with cleavable linker (Moradec, AH-202AF) was incubated with the recombinant anti-CDH17 antibodies to form complex. The formed complexes were then incubated with CDH17 expressing cells. Upon binding to the cell surface CDH17 receptors, the complexes were internalized and the conjugated MMAF was released after lysosomal cleavage of the linker. The released MMAF subsequently inhibited the cell division by blocking the polymerization of tubulin. Briefly, a CDH17 expressing cell line AsPC1 cells were seeded in 96-well plate at 5,000 cells/well and incubated overnight. The recombinant anti-CDH17 antibodies were mixed with the Fab anti-human IgG Fc-MMAF conjugates with cleavable linker at 1:6 ratio (mol/mol) and incubated for 10 min to form the complex. Then, a series dilution of the complexes, from 4.5 pM to 30 nM, were added to each well and the cell viability was measured by Cytation5 imaging system for 48 hours. The dose-response curves were plotted and fitted by GraphPad Prism 9.

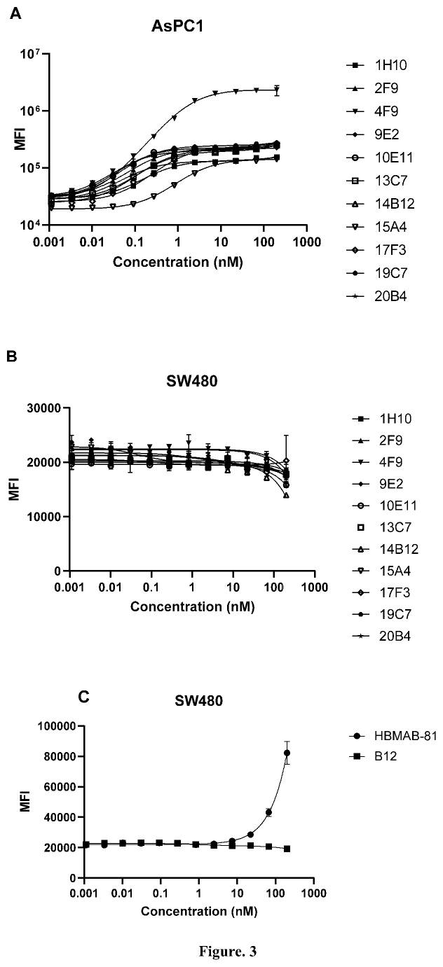

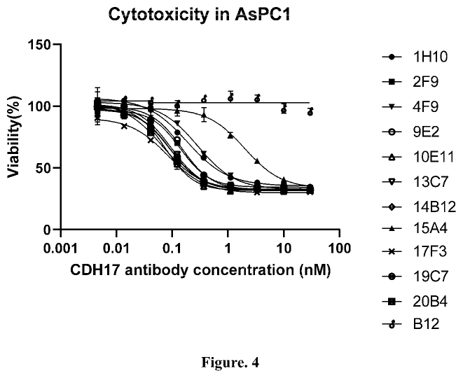

As shown in the FIG. 4 and Table 7, compared to the negative control clone (B12), all the antibody clones showed killing effect in AsPC1 cell line indicating MMAF was delivered into the cells efficiently.

TABLE 7
IC₅₀ of indirect killing assay to evaluate the internalization
of recombinant anti-CDH17 antibodies into AsPC1 cells.

| Clone ID | IC₅₀ (nM) |
|---|---|
| 1H10 | 0.19 |
| 2F9 | 0.14 |
| 4F9 | 0.29 |
| 9E2 | 0.15 |
| 10E11 | 0.08 |
| 13C7 | 0.09 |
| 14B12 | 0.09 |
| 15A4 | 1.99 |
| 17F3 | 0.08 |
| 19C7 | 0.09 |
| 20B4 | 0.07 |

### Example 6 Binding Activities of Anti-CDH17 Clones to Cross-Species CDH17 and Homologs by Octet Characterization

The affinity between CDH17 antibodies and CDH17 antigens and homologues was determined by Octet (Octet Red 384) instrument. Anti-hIgG Fc Capture (AHC) Biosensors were selected, and the sensors were balanced with buffer solution for 10 min. Subsequently, sensors were dipped into the wells containing 1 g/ml of CDH17 lead antibodies to load onto the probes. The excess unbound antibodies were washed off. The antigen binding was performed in the wells containing serially diluted human CDH17-His tag (Sino biological, catalog number 11360-H08H), cynomolgus CDH17-His tag (Acro Biosystems, catalog number CA7-C52H4), homologs human CDH6-His tag (Acro Biosystems, catalog number CA6-H5229) and human CDH16-His tag (Sino biological, catalog number 10915-H08H), with concentration ranging from 200 nM to 3.1 nM, for 10 min. Then probes were dipped into wells containing renewed buffer to initiate the dissociation for another 30 min.

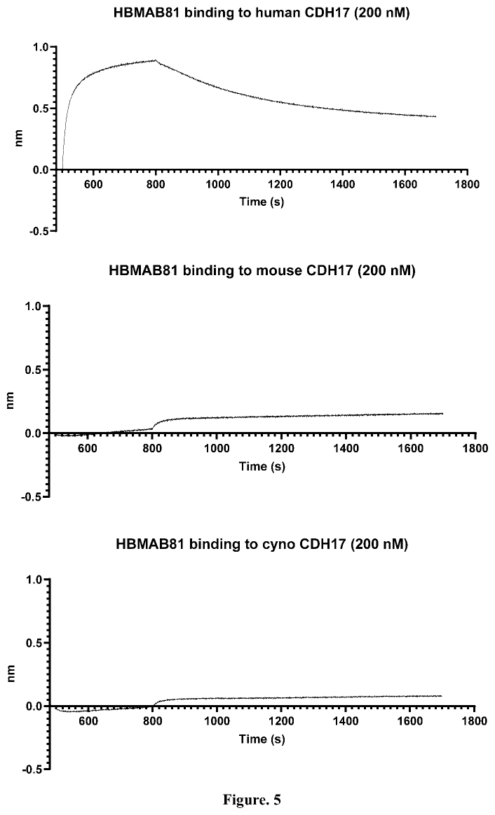

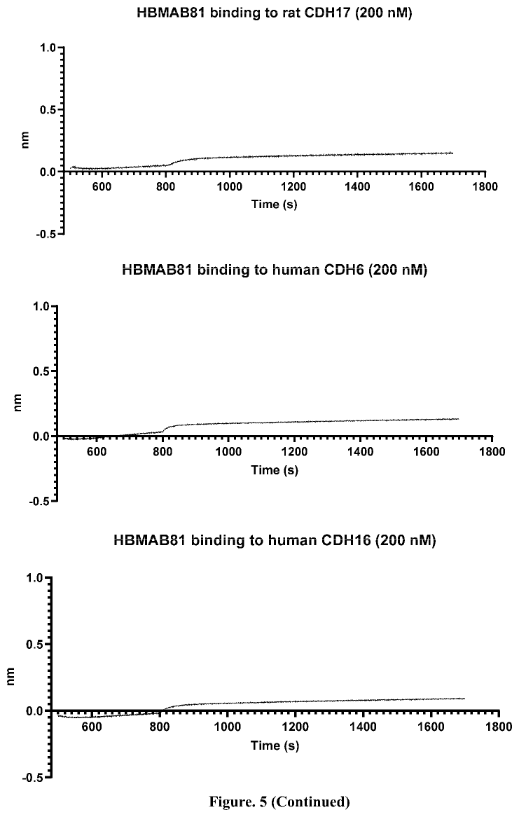

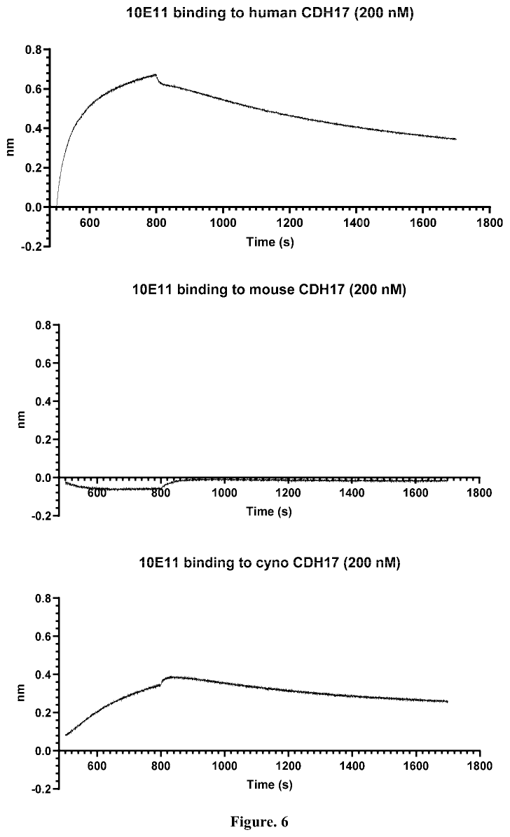

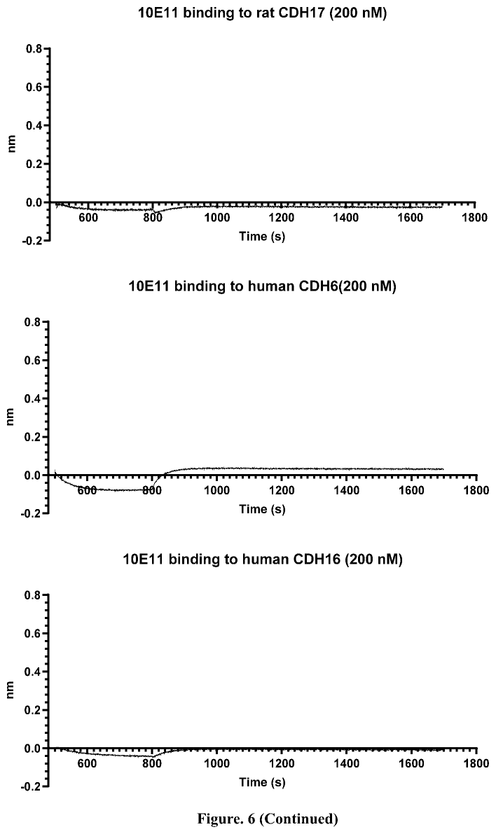

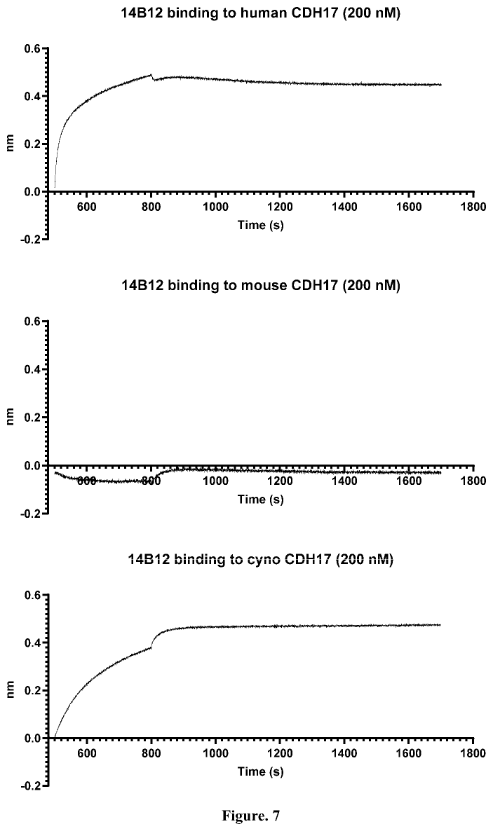

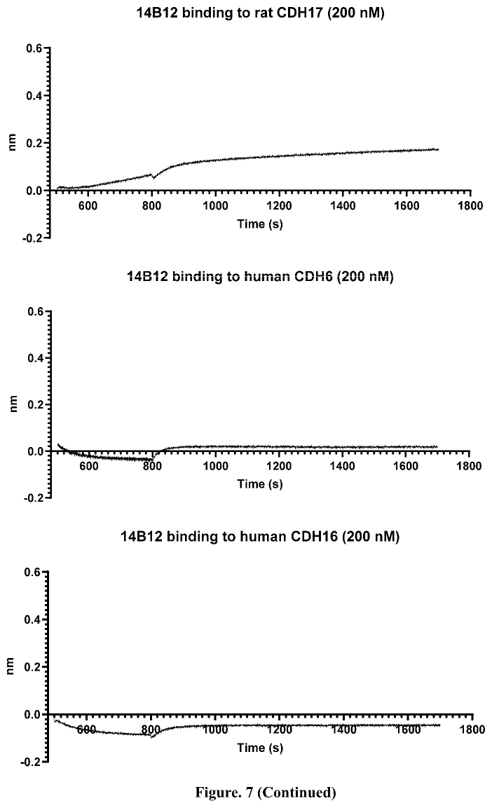

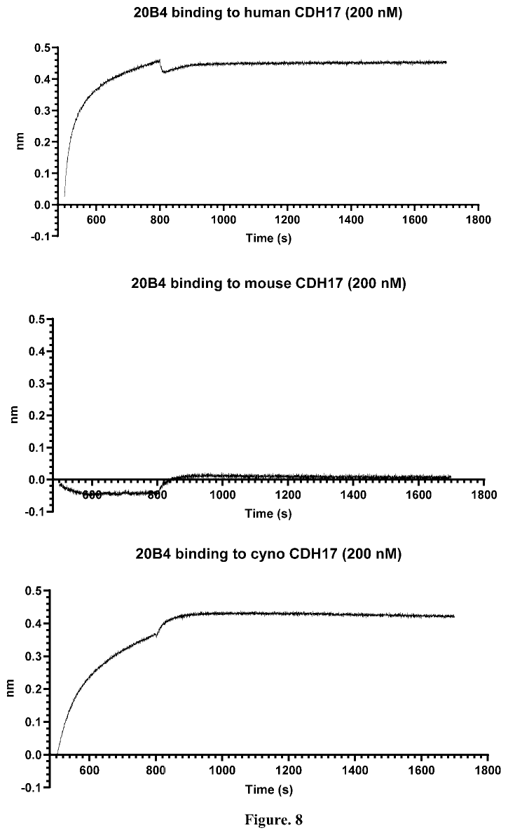

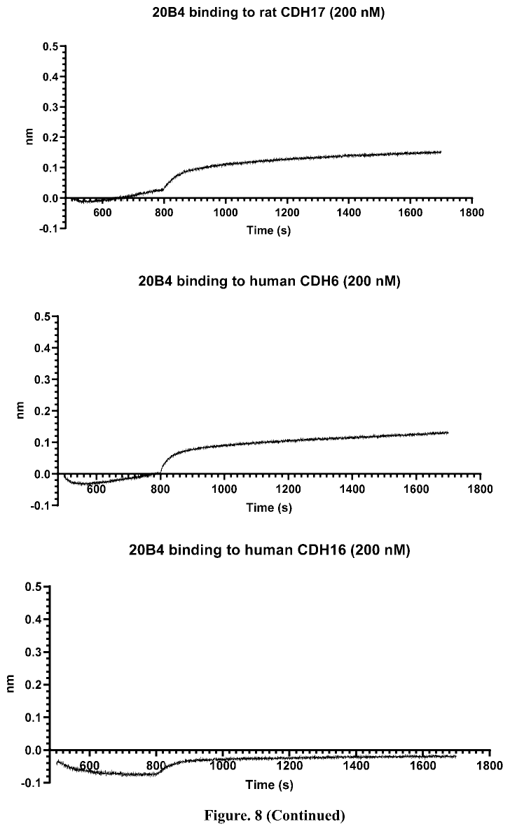

The results showed that 10E11, 14B12 and 20B4 bound to both human and cynomolgus CDH17 proteins, while HBMAB81 only bound to human CDH17 protein (FIG. 5 to FIG. 8, Table 8).

TABLE 8
Binding activities of anti-CDH17 clones
to cross-species CDH17 and homologs.

| | | Antigen proteins | | | |
|---|---|---|---|---|---|
| Clone ID | | hCDH17 | cCDH17 | hCDH6 | hCDH16 |
| HBMAB81 | K_{D} (nM) | 4.0 | N/A | N/A | N/A |
| | Kₒₙ (1/Ms) | 3.4E+05 | N/A | N/A | N/A |
| | K_{off} (1/s) | 1.36E−03 | N/A | N/A | N/A |
| 10E11 | K_{D} (nM) | 14.2 | 15.0 | N/A | N/A |
| | Kₒₙ (1/Ms) | 9.75E+04 | 8.00E+04 | N/A | N/A |
| | K_{off} (1/s) | 1.38E−03 | 1.20E−03 | N/A | N/A |
| 14B12 | K_{D} (nM) | 2.2 | 2.5 | N/A | N/A |
| | Kₒₙ (1/Ms) | 2.3E+05 | 6.2E+05 | N/A | N/A |
| | K_{off} (1/s) | 5.07E−04 | 1.6E−04 | N/A | N/A |
| 20B4 | K_{D} (nM) | 1.1 | 3.0 | N/A | N/A |
| | Kₒₙ (1/Ms) | 1.62E+05 | 9.80E+04 | N/A | N/A |
| | K_{off} (1/s) | 1.87E−04 | 2.94E−04 | N/A | N/A |

### Example 7 Binding Competition

20B4 and HBMAB81 mAbs were labeled with Alexa Flour 647 according to the manufacturer's instruction (ThermoFisher #A20186). In the binding competition assay, 20B4-AF647 and HBMAB81-AF647 were fixed at their EC80 concentration 1.51 nM and 0.97 nM, respectively. The 20B4 and HBMAB81 ranging from 0.03 nM to 1800 nM were first mixed with 1.51 nM 20B4-AF647 or 0.97 nM HBMAB81-647, then incubated with AsPC1 cells on ice for 1 h. Cells were washed twice with cell staining buffer. The samples were analyzed using iQue3 to measure the median fluorescence intensity using corresponding channels.

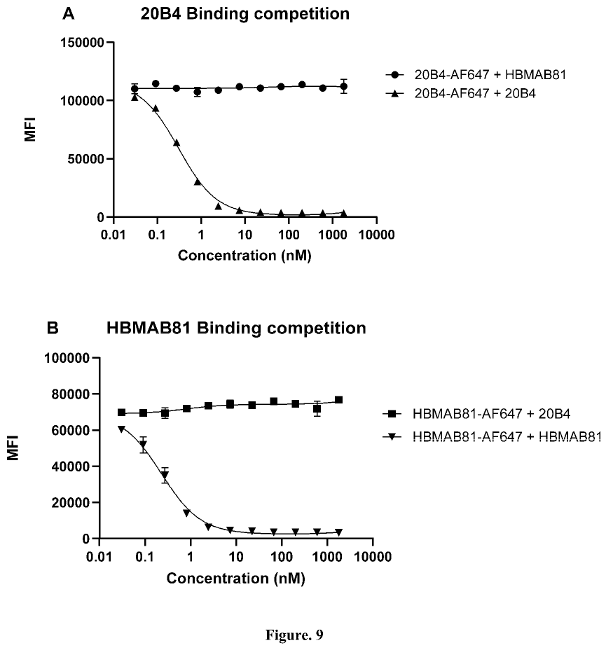

The result was disclosed in FIG. 9. The groups “20B4-AF647+HBMAB81” and “HBMAB-AF647+20B4” didn't show binding inhibition. This indicated that 20B4 and HBMAB81 do not bind to the same or similar epitope.

Although specific embodiments of the invention have been described in detail, those skilled in the art will understand that various modifications and changes may be made to the details in accordance with all the published teachings, which are also included in the scope of the invention. The protection scope of the invention is defined by the appended claims and any equivalents thereof.

1. (canceled)

2. An anti-CDH17 antibody or the antigen-binding fragment thereof comprising:
a heavy chain variable region comprising HCDR1, HCDR2 and HCDR3 having the amino acid sequences as shown in SEQ ID NO: 10, SEQ ID NO: 21 and SEQ ID NO: 31, respectively; and a light chain variable region comprising LCDR1, LCDR2 and LCDR3 having the amino acid sequences as shown in SEQ ID NO: 42, SEQ ID NO: 47 and SEQ ID NO: 54, respectively;
or
a heavy chain variable region comprising HCDR1, HCDR2 and HCDR3 having the amino acid sequences as shown in SEQ ID NO: 07, SEQ ID NO: 17 and SEQ ID NO: 28, respectively; and a light chain variable region comprising LCDR1, LCDR2 and LCDR3 having the amino acid sequences as shown in SEQ ID NO: 38, SEQ ID NO: 45 and SEQ ID NO: 53, respectively;
or
a heavy chain variable region comprising HCDR1, HCDR2 and HCDR3 having the amino acid sequences as shown in SEQ ID NO: 05, SEQ ID NO: 15 and SEQ ID NO: 26, respectively; and a light chain variable region comprising LCDR1, LCDR2 and LCDR3 having the amino acid sequences as shown in SEQ ID NO: 36, SEQ ID NO: 43 and SEQ ID NO: 51, respectively.

3. The anti-CDH17 antibody or the antigen-binding fragment thereof according to claim 1, wherein the antibody or antigen-binding fragment thereof is a monoclonal antibody or antigen-binding fragment thereof, a polyclonal antibody or antigen-binding fragment thereof, a multi-specific antibody or antigen-binding fragment thereof, a murine antibody or antigen-binding fragment thereof, a chimeric antibody or antigen-binding fragment thereof, a humanized antibody or antigen-binding fragment thereof, a recombinant antibody or antigen-binding fragment thereof, or a human antibody or antigen-binding fragment thereof.

4. The anti-CDH17 antibody or the antigen-binding fragment thereof according to claim 2, wherein the antibody or antigen-binding fragment thereof comprises: a heavy chain variable region comprising an amino acid sequence selected from the group consisting of: SEQ ID NOs: 65, 61, 59, or sequence having at least 80% sequence identity therewith; and/or a light chain variable region comprising an amino acid sequence selected from the group consisting of: SEQ ID NOs: 76, 72, 70, or sequence having at least 80%.

5. The anti-CDH17 antibody or the antigen-binding fragment thereof according to claim 4, wherein the antibody or antigen-binding fragment thereof comprises:
the heavy chain variable region having the amino acid sequence as shown in SEQ ID NO: 65, or having the amino acid sequence of at least 80% sequence identity therewith; and/or the light chain variable region having the amino acid sequence as shown in SEQ ID NO: 76, or having the amino acid sequence of at least 80% sequence identity therewith; or
the heavy chain variable region having the amino acid sequence as shown in SEQ ID NO: 61, or having the amino acid sequence of at least 80% sequence identity therewith; and/or the light chain variable region having the amino acid sequence as shown in SEQ ID NO: 72, or having the amino acid sequence of at least 80% sequence identity therewith; or
the heavy chain variable region having the amino acid sequence as shown in SEQ ID NO: 59, or having the amino acid sequence of at least 80% sequence identity therewith; and/or the light chain variable region having the amino acid sequence as shown in SEQ ID NO: 70, or having the amino acid sequence of at least 80% sequence identity therewith.

6. (canceled)

7. The anti-CDH17 antibody or antigen-binding fragment thereof according to claim 5, wherein the antibody or antigen-binding fragment thereof comprises:
the heavy chain variable region having the amino acid sequence as shown in SEQ ID NO: 65; and the light chain variable region having the amino acid sequence as shown in SEQ ID NO: 76;
or
the heavy chain variable region having the amino acid sequence as shown in SEQ ID NO: 61; and the light chain variable region having the amino acid sequence as shown in SEQ ID NO: 72;
or
the heavy chain variable region having the amino acid sequence as shown in SEQ ID NO: 59; and the light chain variable region having the amino acid sequence as shown in SEQ ID NO: 70.

8. The anti-CDH17 antibody or the antigen-binding fragment thereof according to claim 2 further comprising human antibody constant regions;
wherein:
a heavy chain constant region of the human antibody constant regions is selected from constant regions of human IgG1, IgG2, IgG3 and IgG4 and conventional variants thereof, and the light chain constant region of the human antibody constant regions is selected from κ and λ chain constant regions of human antibody and conventional variants thereof,
or
full-length antibody comprises a human antibody heavy chain constant region of SEQ ID NO: 99 or having the amino acid sequence of at least 80% sequence identity therewith, and a human light chain constant region of SEQ ID NO: 100 or having the amino acid sequence of at least 80% sequence identity therewith;
or
a full-length antibody comprises a human antibody heavy chain constant region of SEQ ID NO: 99 and a human light chain constant region of SEQ ID NO: 100.

9. The anti-CDH17 antibody or the antigen-binding fragment thereof according to claim 2, wherein the antibody or antigen-binding fragment thereof comprises:
a heavy chain having the amino acid sequence as shown in SEQ ID NO: 97 or having the amino acid sequence of at least 80% sequence identity therewith, and a light chain having the amino acid sequence as shown in SEQ ID NO: 98 or having the amino acid sequence of at least 80% sequence identity therewith;
or
a heavy chain having the amino acid sequence as shown in SEQ ID NO: 89 or having the amino acid sequence of at least 80%, 85%, 90%, 95% or 99% sequence identity therewith, and a light chain having the amino acid sequence as shown in SEQ ID NO: 90 or having the amino acid sequence of at least 80% sequence identity therewith;
or
a heavy chain having the amino acid sequence as shown in SEQ ID NO: 85 or having the amino acid sequence of at least 80% sequence identity therewith, and a light chain having the amino acid sequence as shown in SEQ ID NO: 86 or having the amino acid sequence of at least 80% sequence identity therewith.

10. The anti-CDH17 antibody or the antigen-binding fragment thereof according to claim 9, wherein the antibody or antigen-binding fragment thereof comprises:
a heavy chain having the amino acid sequence as shown in SEQ ID NO: 97 and a light chain having the amino acid sequence as shown in SEQ ID NO: 98;
or
a heavy chain having the amino acid sequence as shown in SEQ ID NO: 89 and a light chain having the amino acid sequence as shown in SEQ ID NO: 90;
or
a heavy chain having the amino acid sequence as shown in SEQ ID NO: 85 and a light chain having the amino acid sequence as shown in SEQ ID NO: 86.

11. The anti-CDH17 antibody or the antigen-binding fragment thereof according to claim 2, wherein the antigen-binding fragment is selected from the group consisting of Fab, Fab′, F(ab′)2, variable fragment (Fv), single chain variable fragment (scFv), dimerized domain V (diabody), disulfide stabilized Fv (dsFv), and CDR-containing peptides.

12. The anti-CDH17 antibody or the antigen-binding fragment thereof according to claim 2, wherein;
the antibody or antigen-binding fragment binds to both human and cynomolgus CDH17 with a K_{D}≤1000 nM;
or
the antibody or antigen-binding fragment competes with antibody 20B4 for binding to human CDH17.

13. (canceled)

14. An isolated anti-CDH17 antibody or antigen-binding fragment thereof wherein the antibody competes with the antibody or antigen binding fragment according to claim 2 for binding to human CDH17.

15. An isolated nucleic acid molecule encoding the anti-CDH17 antibody or the antigen-binding fragment thereof according to claim 14.

16. A recombinant vector comprising the isolated nucleic acid molecule according to claim 15.

17. A host cell comprising the recombinant vector according to claim 16, wherein the host cell is a prokaryotic cell or a eukaryotic cell.

18. A therapeutic conjugate comprising the anti-CDH17 antibody or the antigen-binding fragment thereof according to claim 2, wherein the antibody or the antigen-binding fragment thereof is conjugated with one or more therapeutic agents.

19. A diagnostic conjugate comprising the anti-CDH17 antibody or the antigen-binding fragment thereof according to claim 2, wherein the antibody or the antigen-binding fragment thereof is conjugated with one or more diagnostic agents.

20. A bispecific antibody comprising a first binding domain that specifically binds to CDH17, and a second binding domain that specifically binds to a second target protein, wherein the first binding domain is the anti-CDH17 antibody or antigen-binding fragment according to claim 2.

21-22. (canceled)

23. A method for diagnosing a disease related to a human CDH17 positive cell, wherein the method comprises contacting a sample with the anti-CDH17 antibody or the antigen-binding fragment thereof of claim 2.

24. A pharmaceutical composition comprising: a therapeutically effective amount or prophylactically effective amount of the anti-CDH17 antibody or the antigen-binding fragment thereof according to claim 2, and one or more pharmaceutically acceptable carriers, diluents, or excipients.

25. A method of treatment or prevention of a CDH17-mediated disease or disorder, the method comprising administering a therapeutically effective amount or prophylactically effective amount of the anti-CDH17 antibody or the antigen-binding fragment according to claim 2, to a subject in need of treatment or prevention of the CDH17-mediated disease or disorder, wherein the disease or disorder is cancer.

26. (canceled)


SEQUENCE LISTING (100 sequences)

SEQ ID NO: 1 - protein, 5 aa, Homo sapiens
SYGMH

SEQ ID NO: 2 - protein, 5 aa, Homo sapiens
DYGMS

SEQ ID NO: 3 - protein, 6 aa, Homo sapiens
SSNWWS

SEQ ID NO: 4 - protein, 5 aa, Homo sapiens
NYGMS

SEQ ID NO: 5 - protein, 5 aa, Homo sapiens
SYWIG

SEQ ID NO: 6 - protein, 7 aa, Homo sapiens
TSGVGVG

SEQ ID NO: 7 - protein, 5 aa, Homo sapiens
TYYRS

SEQ ID NO: 8 - protein, 5 aa, Homo sapiens
SYSLK

SEQ ID NO: 9 - protein, 7 aa, Homo sapiens
TGGVGVG

SEQ ID NO: 10 - protein, 5 aa, Homo sapiens
EVFMH

SEQ ID NO: 11 - protein, 17 aa, Homo sapiens
FIWYDGSNKNYADSVKG

SEQ ID NO: 12 - protein, 17 aa, Homo sapiens
GLSWNGGNTYYADSVKG

SEQ ID NO: 13 - protein, 16 aa, Homo sapiens
EIYHSGSTSYNPSLKS

SEQ ID NO: 14 - protein, 17 aa, Homo sapiens
GINWNGGSTYYADSVRG

SEQ ID NO: 15 - protein, 17 aa, Homo sapiens
IIYPGDSDTRYSPSFQG

SEQ ID NO: 16 - protein, 16 aa, Homo sapiens
LIYWNDDKRYRPSLKN

SEQ ID NO: 17 - protein, 16 aa, Homo sapiens
YIYYSGNTNYNPSLKS

SEQ ID NO: 18 - protein, 17 aa, Homo sapiens
FVWYDGSNQNYADSVKG

SEQ ID NO: 19 - protein, 17 aa, Homo sapiens
SISSSSGYIYYADSVKG

SEQ ID NO: 20 - protein, 16 aa, Homo sapiens
LIYWNDDKRYSPSLKS

SEQ ID NO: 21 - protein, 17 aa, Homo sapiens
GFDPEDGETIYAQKFQG

SEQ ID NO: 22 - protein, 12 aa, Homo sapiens
DRYSSGWDVFDY

SEQ ID NO: 23 - protein, 13 aa, Homo sapiens
GSRYNWNYDAFDI

SEQ ID NO: 24 - protein, 9 aa, Homo sapiens
NDWGFPLDC

SEQ ID NO: 25 - protein, 13 aa, Homo sapiens
GSRYNWSYDAFDI

SEQ ID NO: 26 - protein, 9 aa, Homo sapiens
RGWVNYFDY

SEQ ID NO: 27 - protein, 13 aa, Homo sapiens
YGVTAPFYNWFDP

SEQ ID NO: 28 - protein, 10 aa, Homo sapiens
AQYGEEAFDI

SEQ ID NO: 29 - protein, 18 aa, Homo sapiens
AVSTIAARPGYYYYYMDV

SEQ ID NO: 30 - protein, 13 aa, Homo sapiens
SGLAAPFYNWFDP

SEQ ID NO: 31 - protein, 9 aa, Homo sapiens
GWGSRYFDY

SEQ ID NO: 32 - protein, 11 aa, Homo sapiens
RASQDISSWLA

SEQ ID NO: 33 - protein, 11 aa, Homo sapiens
RASQGIRNALG

SEQ ID NO: 34 - protein, 11 aa, Homo sapiens
RASQSISSYLN

SEQ ID NO: 35 - protein, 11 aa, Homo sapiens
RASQGVRNALG

SEQ ID NO: 36 - protein, 11 aa, Homo sapiens
RASQGISNWLG

SEQ ID NO: 37 - protein, 11 aa, Homo sapiens
RASQSITSYLN

SEQ ID NO: 38 - protein, 11 aa, Homo sapiens
RASQSITTYLN

SEQ ID NO: 39 - protein, 11 aa, Homo sapiens
RASQGISSWLA

SEQ ID NO: 40 - protein, 11 aa, Homo sapiens
RASQDISTWFA

SEQ ID NO: 41 - protein, 11 aa, Homo sapiens
RASQSISNYLN

SEQ ID NO: 42 - protein, 17 aa, Homo sapiens
KSSQSVLYSSNNKNYLA

SEQ ID NO: 43 - protein, 7 aa, Homo sapiens
AASSLQS

SEQ ID NO: 44 - protein, 7 aa, Homo sapiens
AASNLQS

SEQ ID NO: 45 - protein, 7 aa, Homo sapiens
AASRLQT

SEQ ID NO: 46 - protein, 7 aa, Homo sapiens
AASTLRS

SEQ ID NO: 47 - protein, 7 aa, Homo sapiens
WASTRES

SEQ ID NO: 48 - protein, 9 aa, Homo sapiens
QQAKSFPPT

SEQ ID NO: 49 - protein, 9 aa, Homo sapiens
LQHNSYPFT

SEQ ID NO: 50 - protein, 9 aa, Homo sapiens
QQSHNIPIT

SEQ ID NO: 51 - protein, 9 aa, Homo sapiens
QQANSFPLT

SEQ ID NO: 52 - protein, 8 aa, Homo sapiens
QRSYSTLT

SEQ ID NO: 53 - protein, 9 aa, Homo sapiens
QQSYNTPPT

SEQ ID NO: 54 - protein, 9 aa, Homo sapiens
HQYYSTPLT

SEQ ID NO: 55 - protein, 121 aa, Homo sapiens
QVQLVESGGGVVQPGRSLRLSCVASGFTFSSYGMHWVRQAPGKGLEWVTFIWYDGSNKNYADSVKGRFTISRDNSKNTLNLQMNSLRVEDTAVYYCARDRYSSGWDVFDYWGQGTLVTVSS

SEQ ID NO: 56 - protein, 122 aa, Homo sapiens
EVQLVESGGGVVRPGGSLRLSCAASGFTFDDYGMSWVRQAPGKGLEWVSGLSWNGGNTYYADSVKGRFTISRDNAKNSLYLQMNSLRAEDTALYYCARGSRYNWNYDAFDIWGQGTLVTVSS

SEQ ID NO: 57 - protein, 118 aa, Homo sapiens
QVQLQESGPGLVKPSGTLSLTCAVSGGSISSSNWWSWVRQPPGKGLEWIGEIYHSGSTSYNPSLKSRVTISVDKSKNQFSLKLSSVTAADTAVYYCARNDWGFPLDCWGQGTLVTVSS

SEQ ID NO: 58 - protein, 122 aa, Homo sapiens
EVQLVESGGGVVRPGGSLRLSCAASGFTFDNYGMSWVRQAPGKGLEWVSGINWNGGSTYYADSVRGRFTISRDNAKNSLYLQMNSLRAEDTALYYCARGSRYNWSYDAFDIWGQGTLVTVSS

SEQ ID NO: 59 - protein, 118 aa, Homo sapiens
EVQLVQSGTEVKKPGESLKISCKDSGYRFSSYWIGWVRQMPGKGLEWMGIIYPGDSDTRYSPSFQGQVTISADKSISTAYLQWRSLKASDTAMYYCARRGWVNYFDYWGQGTLVTVSS

SEQ ID NO: 60 - protein, 123 aa, Homo sapiens
QITLKESGPTLVKPTQTLTLTCTFSGFSLSTSGVGVGWIRQPPGKALEWLALIYWNDDKRYRPSLKNRLTITKDTSKNQVVLTMTNMDPVDTATYYCTHYGVTAPFYNWFDPWGQGTLVTVSS

SEQ ID NO: 61 - protein, 118 aa, Homo sapiens
QVQLQESGPGLVKPSETLSLTCTVSGGSINTYYRSWIRQPPGKGLEWIGYIYYSGNTNYNPSLKSRVTISADTSKNQFSLNLISVTAADTAMYYCARAQYGEEAFDIWGQGTLVTVSS

SEQ ID NO: 62 - protein, 121 aa, Homo sapiens
QVQLVESGGGVVQPGRSLRLSCAASGFNFSSYGMHWVRQAPGKGLEWVAFVWYDGSNQNYADSVKGRFTISRDNSNNTLYLQMNSLRAEDTAVYYCARDRYSSGWDVFDYWGQGTLVTVSS

SEQ ID NO: 63 - protein, 127 aa, Homo sapiens
EVQLVESGGGLVKSGGSLRLSCAASGFTFSSYSLKWVRQAPGKGLEWVSSISSSSGYIYYADSVKGRFTISRDNAKNSLYLQMNSLRAEDTAMYYCARAVSTIAARPGYYYYYMDVWGKGTLVTVSS

SEQ ID NO: 64 - protein, 123 aa, Homo sapiens
QITLKESGPTLVKPTQTLTLTCTFSGISLSTGGVGVGWIRQPPGKALEWLSLIYWNDDKRYSPSLKSRLTITKDSSKNQVVLTMTNMDPVDTATYYCAHSGLAAPFYNWFDPWGQGTLVTVSS

SEQ ID NO: 65 - protein, 118 aa, Homo sapiens
QVQLVQSGAEVKKPGASVKVSCKVSGYTLTEVFMHWVRQAPGKGLEWMGGFDPEDGETIYAQKFQGRVTMTEDTFIDTAYMDLSSLRSEDTAVYYCAIGWGSRYFDYWGQGTLVTVSS

SEQ ID NO: 66 - protein, 107 aa, Homo sapiens
DIQMTQSPSSVSASVGDRVTITCRASQDISSWLAWYQQQPGKAPKLQIYAASSLQSGVPSRFSGSGSGTDFTLTISSLQPEDFATYYCQQAKSFPPTFGQGTKVEIK

SEQ ID NO: 67 - protein, 107 aa, Homo sapiens
DIQMTQSPSSLSASIGDRVTITCRASQGIRNALGWYQQKPGKAPKRLIYAASNLQSGVPSRFSGSGSGTEFTLTISSLQPEDFATYYCLQHNSYPFTFGPGTKVEIK

SEQ ID NO: 68 - protein, 107 aa, Homo sapiens
DIQMTQSPSSLSASVGDRVTITCRASQSISSYLNWYQQKPGKAPKLLIYAASSLQSGVPSRFSGSGSGTHFTLTISSLQPEDFATYYCQQSHNIPITFGQGTRLEIK

SEQ ID NO: 69 - protein, 107 aa, Homo sapiens
DIQMTQSPSSLSASVGDRVTITCRASQGVRNALGWYQQKPGKAPKRLIYAASSLQSGVPSRFSGSGSGTEFTLTISSLQPEDFATYYCLQHNSYPFTFGPGTKVEIK

SEQ ID NO: 70 - protein, 107 aa, Homo sapiens
DIQMTQSPSSVSASVGDRVTITCRASQGISNWLGWYQQKPGKAPKLLIYAASSLQSGVPSRFSGSGSGTDFTLTISSLQPEDFATYYCQQANSFPLTFGGGTKVEIK

SEQ ID NO: 71 - protein, 106 aa, Homo sapiens
DIQMTQSPSSLSASVGDRVTITCRASQSITSYLNWYQQKPGKAPKLLIYAASSLQSGVPSRFSGSGSGTDFTLTISSLQPEDFATYYCQRSYSTLTFGGGTKVEIK

SEQ ID NO: 72 - protein, 107 aa, Homo sapiens
DIQMTQSPSSLSASVGDRVTITCRASQSITTYLNWYQQKPGKAPQLLIFAASRLQTGVPSRFSGSGSGTDFTLTISSLLPGDFATYYCQQSYNTPPTFGGGTKVEIK

SEQ ID NO: 73 - protein, 107 aa, Homo sapiens
DIQMTQSPSSVSASVGDRVTITCRASQGISSWLAWYQQKPGKAPKLLIFAASSLQSGVPSRFSGSGSGTGFTLTISSLQPEDFATYYCQQAKSFPPTFGQGTKVEIK

SEQ ID NO: 74 - protein, 107 aa, Homo sapiens
DIQMTQSPSSVSASIGDRVTITCRASQDISTWFAWYQQKPGKAPKLLIYAASTLRSGVPSRFSGSGSGTDFTLTINSLQPEDFATYYCQQANSFPLTFGGGTKVEIK

SEQ ID NO: 75 - protein, 106 aa, Homo sapiens
DIQMTQSPSSLSASVGDRVTITCRASQSISNYLNWYQLKPGNAPKLLIFAASSLQSGVPSRFSGSGSGTDFTLTISSLQPEDFATYFCQRSYSTLTFGGGTKVEIK

SEQ ID NO: 76 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCHQYYSTPLTFGGGTKVEIK

SEQ ID NO: 77 - protein, 451 aa, synthetic construct
  source 1..451: note = 1H10
QVQLVESGGGVVQPGRSLRLSCVASGFTFSSYGMHWVRQAPGKGLEWVTFIWYDGSNKNYADSVKGRFTISRDNSKNTLNLQMNSLRVEDTAVYYCARDRYSSGWDVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 214 aa, synthetic construct
  source 1..214: note = 1H10
DIQMTQSPSSVSASVGDRVTITCRASQDISSWLAWYQQQPGKAPKLQIYAASSLQSGVPSRFSGSGSGTDFTLTISSLQPEDFATYYCQQAKSFPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 452 aa, synthetic construct
  source 1..452: note = 2F9
EVQLVESGGGVVRPGGSLRLSCAASGFTFDDYGMSWVRQAPGKGLEWVSGLSWNGGNTYYADSVKGRFTISRDNAKNSLYLQMNSLRAEDTALYYCARGSRYNWNYDAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 214 aa, synthetic construct
  source 1..214: note = 2F9
DIQMTQSPSSLSASIGDRVTITCRASQGIRNALGWYQQKPGKAPKRLIYAASNLQSGVPSRFSGSGSGTEFTLTISSLQPEDFATYYCLQHNSYPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 448 aa, synthetic construct
  source 1..448: note = 4F9
QVQLQESGPGLVKPSGTLSLTCAVSGGSISSSNWWSWVRQPPGKGLEWIGEIYHSGSTSYNPSLKSRVTISVDKSKNQFSLKLSSVTAADTAVYYCARNDWGFPLDCWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 214 aa, synthetic construct
  source 1..214: note = 4F9
DIQMTQSPSSLSASVGDRVTITCRASQSISSYLNWYQQKPGKAPKLLIYAASSLQSGVPSRFSGSGSGTHFTLTISSLQPEDFATYYCQQSHNIPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 83 - protein, 452 aa, synthetic construct
  source 1..452: note = 9E2
EVQLVESGGGVVRPGGSLRLSCAASGFTFDNYGMSWVRQAPGKGLEWVSGINWNGGSTYYADSVRGRFTISRDNAKNSLYLQMNSLRAEDTALYYCARGSRYNWSYDAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 214 aa, synthetic construct
  source 1..214: note = 9E2
DIQMTQSPSSLSASVGDRVTITCRASQGVRNALGWYQQKPGKAPKRLIYAASSLQSGVPSRFSGSGSGTEFTLTISSLQPEDFATYYCLQHNSYPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 448 aa, synthetic construct
  source 1..448: note = 10E11
EVQLVQSGTEVKKPGESLKISCKDSGYRFSSYWIGWVRQMPGKGLEWMGIIYPGDSDTRYSPSFQGQVTISADKSISTAYLQWRSLKASDTAMYYCARRGWVNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 214 aa, synthetic construct
  source 1..214: note = 10E11
DIQMTQSPSSVSASVGDRVTITCRASQGISNWLGWYQQKPGKAPKLLIYAASSLQSGVPSRFSGSGSGTDFTLTISSLQPEDFATYYCQQANSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 453 aa, synthetic construct
  source 1..453: note = 13C7
QITLKESGPTLVKPTQTLTLTCTFSGFSLSTSGVGVGWIRQPPGKALEWLALIYWNDDKRYRPSLKNRLTITKDTSKNQVVLTMTNMDPVDTATYYCTHYGVTAPFYNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 213 aa, synthetic construct
  source 1..213: note = 13C7
DIQMTQSPSSLSASVGDRVTITCRASQSITSYLNWYQQKPGKAPKLLIYAASSLQSGVPSRFSGSGSGTDFTLTISSLQPEDFATYYCQRSYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 448 aa, synthetic construct
  source 1..448: note = 14B12
QVQLQESGPGLVKPSETLSLTCTVSGGSINTYYRSWIRQPPGKGLEWIGYIYYSGNTNYNPSLKSRVTISADTSKNQFSLNLISVTAADTAMYYCARAQYGEEAFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 214 aa, synthetic construct
  source 1..214: note = 14B12
DIQMTQSPSSLSASVGDRVTITCRASQSITTYLNWYQQKPGKAPQLLIFAASRLQTGVPSRFSGSGSGTDFTLTISSLLPGDFATYYCQQSYNTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 451 aa, synthetic construct
  source 1..451: note = 15A4
QVQLVESGGGVVQPGRSLRLSCAASGFNFSSYGMHWVRQAPGKGLEWVAFVWYDGSNQNYADSVKGRFTISRDNSNNTLYLQMNSLRAEDTAVYYCARDRYSSGWDVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 214 aa, synthetic construct
  source 1..214: note = 15A4
DIQMTQSPSSVSASVGDRVTITCRASQGISSWLAWYQQKPGKAPKLLIFAASSLQSGVPSRFSGSGSGTGFTLTISSLQPEDFATYYCQQAKSFPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 457 aa, synthetic construct
  source 1..457: note = 17F3
EVQLVESGGGLVKSGGSLRLSCAASGFTFSSYSLKWVRQAPGKGLEWVSSISSSSGYIYYADSVKGRFTISRDNAKNSLYLQMNSLRAEDTAMYYCARAVSTIAARPGYYYYYMDVWGKGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 214 aa, synthetic construct
  source 1..214: note = 17F3
DIQMTQSPSSVSASIGDRVTITCRASQDISTWFAWYQQKPGKAPKLLIYAASTLRSGVPSRFSGSGSGTDFTLTINSLQPEDFATYYCQQANSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 453 aa, synthetic construct
  source 1..453: note = 19C7
QITLKESGPTLVKPTQTLTLTCTFSGISLSTGGVGVGWIRQPPGKALEWLSLIYWNDDKRYSPSLKSRLTITKDSSKNQVVLTMTNMDPVDTATYYCAHSGLAAPFYNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 213 aa, synthetic construct
  source 1..213: note = 19C7
DIQMTQSPSSLSASVGDRVTITCRASQSISNYLNWYQLKPGNAPKLLIFAASSLQSGVPSRFSGSGSGTDFTLTISSLQPEDFATYFCQRSYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 448 aa, synthetic construct
  source 1..448: note = 20B4
QVQLVQSGAEVKKPGASVKVSCKVSGYTLTEVFMHWVRQAPGKGLEWMGGFDPEDGETIYAQKFQGRVTMTEDTFIDTAYMDLSSLRSEDTAVYYCAIGWGSRYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 220 aa, synthetic construct
  source 1..220: note = 20B4
DIVMTQSPDSLAVSLGERATINCKSSQSVLYSSNNKNYLAWYQQKPGQPPKLLIYWASTRESGVPDRFSGSGSGTDFTLTISSLQAEDVAVYYCHQYYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 107 aa, Homo sapiens [sequence omitted]